(19) United States
(12) Patent Application Publication
(10) Pub. No.: US 2003/0064978 A1
(43) Pub. Date: Apr. 3, 2003

(54) Novel compounds

(76) Inventors: Christopher Walpole, Quebec (CA); Hua Yang, Quebec (CA)

Correspondence Address: Michael A Sanzo, Pillsbury Winthrop, Ninth Floor, 1100 New York Avenue NW, Washington, DC, 20005-3918, US

(21) Appl. No.: 10/203,297

(22) PCT Filed: Feb. 9, 2001
(86) PCT No.: PCT/SE01/00273

(30) Foreign Application Priority Data
Feb. 14, 2000 (SE) 0000477-0

(51) Int. Cl. C07D 487/10; A61K 31/551; A61K 31/55; A61K 31/541; A61K 31/5377; A61K 31/519

(52) U.S. Cl. 514/212.02; 514/218; 514/228.5; 514/232.8; 514/267; 540/543; 544/6; 544/70; 544/231

(57) ABSTRACT

There are provided novel compounds of formula (I) wherein R¹, R², R³, R⁴ and R⁵ are as defined in the Specification and optical isomers, racemates and tautomers thereof and pharmaceutically acceptable salts thereof; together with processes for their preparation, compositions containing them and their use in therapy. The compounds are inhibitors of the enzyme nitric oxide synthase.

### FIELD OF THE INVENTION

The present invention is directed to novel fluoropiperidine spirocycle derivatives that inhibit the inducible isozyme of nitric oxide synthase, processes for their preparation and certain intermediates used in said processes. In addition, the invention is directed to pharmaceutical compositions containing said novel compounds and to the use of such compositions in the treatment of a variety of medical conditions, particularly pain.

### BACKGROUND OF THE INVENTION

In mammalian cells, nitric oxide is generated from the guanidino group of L-arginine upon its conversion into citrulline, a reaction that is catalysed by the enzyme nitric oxide synthase (Moncada el al., Pharm. Rev. 43:109-142 (1991); Langrehr el al., J. Clin. Invest. 90:679-683 (1992)). Nitric oxide synthase (NOS) occurs in three distinct isoforms (Kerwin et al, Med. Res. Rev. 14:23 (1994)). Two of the isoforms are produced constitutively in endothelial cells (eNOS) and in brain cells (bNOS; also known as neuronal NOS, nNOS) respectively. The expression of the third isoform (iNOS) is induced in a variety of different cells in response to endotoxins or cytokines. Nitric oxide generated as a result of iNOS activity appears, inter alia, to protect the host by contributing to the killing of bacteria, fungi and tumour cells.

Excessive cellular nitric oxide generated from iNOS plays an important role in the pathogenesis of many diseases. In particular, nitric oxide appears to contribute to inflammatory and autoimmune diseases such as rheumatoid arthritis, osteoarthritis, inflammatory bowel disease, irritable bowel disease and multiple sclerosis (McInnes et al. J. Exp. Med. 184:1519-1524 (1994); Sakurai et al., J Clin. invest. 96:2357-2363 (1994)). There is evidence that inhibitors of nitric oxide synthase may be useful in the treatment of cardiovascular ischaemia, diabetes, congestive heart failure, atherosclerosis, migraine, asthma, cerebral ischaemia, Parkinson's disease, Alzheimer's disease and in the alleviation of pain (Kerwin el al., J. Med. Chem. 38:44343-4362 (1995); Knowles et al., Biochem. J. 298:249-258 (1994)). In addition, NOS inhibitors may be useful in combination with cytokines and as an adjuvant to immunosuppression during organ transplantation (Moncada et al., FASEB J. 9:1319-1330 (1995); Kilborn et al., Crit. Care Med. 23:1018-1024 (1995)).

Given the large number of diseases affected by excessive levels of nitric oxide, it is not surprising that many attempts have been made to develop inhibitors of NOS. Inhibitors with improved therapeutic properties would represent a clear advance in clinical medicine.

WO 97/14686 discloses novel compounds including, in one embodiment, compounds of generic structure

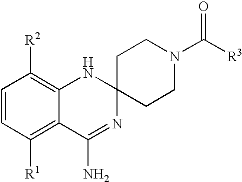

wherein R¹ and R² represent, inter alia, hydrogen, alkyl C1 to 6 or halogen; and R³ represents a variety of cyclic and acyclic moieties. The compounds have nitric oxide synthase inhibitory activity.

It has now surprisingly been found that certain similar compounds wherein the spiro-piperidine ring is substituted by fluorine, and which therefore are not within the generic scope of WO 97/14686, possess unexpectedly advantageous properties. Such compounds, which are useful in therapy, particularly in the treatment of pain are the subject of the present application.

### DISCLOSURE OF THE INVENTION

In a first aspect, the invention is directed to novel compounds having a structure according to general formula (1):

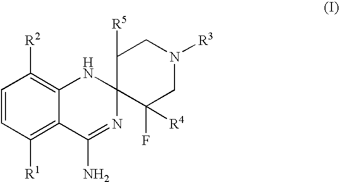

in which:

R¹ represents H, F or Cl;

R² represents H, F or CH₃;

R³ is selected from the group consisting of:

a) H; or

b) —CO—X wherein X represents:

i) a C6 to C10 aromatic ring, optionally substituted by one or more substituents selected independently from CN, Cl, F, Br, I, CF₃, OCF₃, C₁-C₃ alkyl and C₁-C₃ alkoxy;

ii) a heteroaromatic ring having from 5 to 10 ring atoms where at least one ring atom is a heteroatom selected from O, N or S; and wherein said ring is optionally substituted by one or more substituents selected independently from CN, Cl, F, Br, I, CF₃, OCF₃, C₁-C₃ alkyl and C₁-C₃ alkoxy; or

iii) C₁-C₆ alkoxy or —O—(CH₂)ₙ-phenyl, wherein n represents an integer 0 to 3;

and either both R⁴ and R⁵ represent H; or R⁴ represents H and R⁵ represents F, or R⁴ represents F and R⁵ represents H;

and diastereomers, enantiomers, racemates and tautomers thereof and pharmaceutically acceptable salts thereof.

Preferably, both R⁴ and R⁵ represent H. In this case, the compounds of formula (I) exist as pairs of racemic diastereoisomers (diastereomers) which may be conveniently separated by normal or reverse phase chromatography on silica gel or C-18 matrix. These diastereomers differ in the relative orientation of the fluorine atom in the 3-position of the piperidine ring and the amidine nitrogen atom in the 4-position of the piperidine ring (cis or trans relationship).

As used herein, the expression “cis” refers to a compound of general formula (IA) wherein the fluoro substituent is on the same side of the piperidine ring as the nitroren atom of the amidine group:

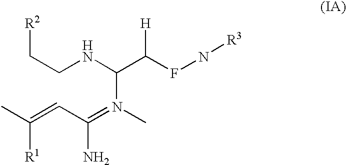

As used herein, the expression “trans” refers to a compound of general formula (IB) wherein the fluoro substituent is on the opposite side of the piperidine ring, to the nitrogen atom of the amidine group:

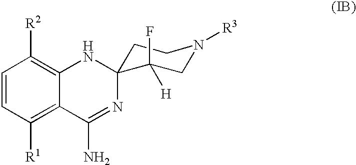

Each diastereomer (IA) or (IB) may be further separated into two constituent enantiomers by methods such as chiral HPLC. Unless otherwise indicated, all structures disclosed and discussed herein are intended to encompass all diastereoisomeric and enantiomeric forms.

Preferably R¹ and R² independently represent H or F.

When R⁴ and R⁵ both represent H, and R¹ and R² both represent F, cis isomers are preferred.

When R⁴ and R⁵ both represent H, and R¹ represents F and R² represents H, trans isomers are preferred.

In other preferred embodiments, R is —CO—X, and X is selected from the group consisting of:

i) phenyl optionally substituted with CN, Cl, F, Br or C₁-C₃ alkyl;

ii) a five or six membered heteroaromatic ring incorporating one or two heteroatoms selected from O, N and S, and wherein said ring is optionally substituted with CN, Cl, F, Br or C₁-C₃ alkyl; or

iii) —O—(CH₂)ₙ-phenyl, wherein n represents an integer 0 to 3.

More preferably, R³ is —CO—X, and X is selected from the group consisting of:

phenyl, furyl, thienyl, pyridyl, oxazolyl or pyrazinyl, optionally substituted with CN, CH₃ or halogen.

Even more preferably, R³ is —CO—X, and X is selected from the group consisting of:

phenyl, furyl, thienyl or pyridyl optionally substituted with CN or Cl.

Particular compounds of the invention include:

cis-1-(4-cyanobenzoyl)-3-fluorospiro[piperidine-4,2′(1′H)-quinazoline]-4′-amine;

trans-1-(4-cyanobenzoyl)-3-fluorospiro[piperidine-4,2′(1′H)-quinazoline]-4′-amine;

cis-1-(4-chlorobenzoyl)-3-fluorospiro[piperidine-4,2′(1′H)-quinazoline]-4′-amine;

trans-1-(4-chlorobenzoyl)-3-fluorospiro[piperidine-4,2′(1′H)-quinazoline]-4′-amine;

cis-1-(6-cyano-3-pyridylcarbonyl)-3-fluorospiro[piperidine-4,2′(1′H)-quinazoline]-4′-amine;

trans-1-(6-cyano-3-pyridylcarbonyl)-3-fluorospiro[piperidine-4,2′(1′H)-quinazoline]-4′-amine;

cis-3-fluoro-1-(4-methylbenzoyl)-spiro[piperidine-4,1′(1′H)-quinazoline]-4′-amine;

trans-3-fluoro-1-(4-methylbenzoyl)-spiro[pipendine-4,2′(1′H)-quinazoline]-4′-amine;

cis-3-fluoro-1-(2-furylcarbonyl)-spiro[piperidine-4,2′(1′H)-quinazoline]-4′-amine:

trans-3-fluoro-1-(2-furylcarbonyl)-spiro[piperidine-4,2′(1′H)-quinazoline]-4′-amine;

cis-3-fluoro-1-(2-thienylcarbonyl)-spiro[piperidine-4,2′(1′H)-quinazoline]-4′-amine;

trans-3-fluoro-1-(2-thienylcarbonyl)-spiro[piperidine-4,2′(1′H)-quinazoline]-4′-amine;

cis-3,5′-difluoro-1-(2-thienylcarbonyl)-spiro[piperidine-4,2′(1′H)-quinazoline]-4′-amine;

trans-3,5′-difluoro-1-(2-thienylcarbonyl)-spiro[piperidine-4,2′(1′H)-quinazoline]-4′-amine;

cis-3,5′-difluoro-1-(4-chlorobenzoyl)-spiro[piperidine-4,2′(1′H)-quinazoline]-4′-amine;

trans-3,5′-difluoro-1-(4-chlorobenzoyl)-spiro[piperidine-4,2′(1′H)-quinazoline]-4′-amine;

cis-3,5′-difluoro-1-(4-cyanobenzoyl)-spiro[piperidine-4,2′(1′H)-quinazoline]-4′-amine;

trans-3,5′-difluoro-1-(4-cyanobenzoyl)-spiro[piperidine-4,2′(1′H)-quinazoline]-4′-amine;

cis-3,5′-difluoro-1-(2-furylcarbonyl)-spiro[piperidine-4,2′(1′H)-quinazoline]-4′-amine:

trans-3,5′-difluoro-1-(2-furylcarbonyl)-spiro[piperidine-4,2′(1′H)-quinazoline]-4′-amine;

cis-3,5′-difluoro-1-(6-cyano-3-pyridylcarbonyl)-spiro[piperidine-4,2′(1′H)-quinazoline]-4′-amine;

trans-3,5′-difluoro-1-(6-cyano-3-pyridylcarbonyl)-spiro[piperidine-4,2′(1′H)-quinazoline]-4′-amine;

cis-3,5′-difluoro-1-(4-methylbenzoyl)-spiro[piperidine-4,2′(1′H)-quinazoline]-4′-amine;

trans-3,5′-difluoro-1-(4-methylbenzoyl)-spiro[piperidine-4,2′(1′H)-quinazoline]-4′-amine;

cis-1-(6-cyano-3-pyridylcarbonyl)-3,5′,8′-trifluorospiro[piperidine-4,2′(1′H)-quinazoline]-4′-amine;

(−)-(3S,2′R)-1-(6-cyano-3-pyridylcarbonyl)-3,5′,8′-trifluorospiro[pipenidine-4,2′(1′H)-quinazoline]-4′-amine;

(+)-(3R,2′S)-1-(6-cyano-3-pyridylcarbonyl)-3,5′,8′-trifluorospiro[piperidine-4,2′(1′H)-quinazoline]-4′-amine;

trans-1-(6-cyano-3-pyridylcarbonyl)-3,5′,8′-trifluorospiro[piperidine-4,2′(1′H)-quinazoline]-4′-amine;

(−)-(3S,2′S)-1-(6-cyano-3-pyridylcarbonyl)-3,5′,8′-trifluorospiro[piperidine-4,2′(1′H)-quinazoline]-4′-amine;

(+)-(3R,2′R)-1-(6-cyano-3-pyridylcarbonyl)-3,5′,8′-trifluorospiro[piperidine-4,2′(1′H)-quinazoline]-4′-amine;

cis-1-(4-chlorobenzoyl)-3,5′,8′-trifluorospiro[piperidine-4,2′(1′H)-quinazoline]-4′-amine;

trans-1-(4-chlorobenzoyl)-3,5′,8′-trifluorospiro[piperidine-4,2′(1′H)-quinazoline]-4′-amine;

benzyl cis-4′-amino-3,5′,8′-trifluorospiro[piperidine-4,2′(1′H)-quinazoilne]-1-carboxylate;

benzyl trans-4′-amino-3,5′,8′-trifluorospiro[piperidine-4,2′(1′H)-quinazoline]-1-carboxylate;

cis-3,5′,8′-trifluorospiro[piperidine-4,2′(1′H)-quinazoline]-4′-amine:

trans-3,5′,8′-trifluorospiro[piperidine-4,2′(1′H)-quinazoline]-4′-amine;

cis-1-(4-cyanobenzoyl)-3,5′,8′-trifluorospiro[piperidine-4,2′(1′H)-quinazoline]-4′-amine;

trans-1-(4-cyanobenzoyl)-3,5′,8′-trifluorospiro[piperidine-4,2′(1′H)-quinazoline]-4′-amine;

cis-1-(2-furylcarbonyl)-3,5′,8′-trifluorospiro[piperidine-4,2′(1′H)-quinazoline]-4′-amine;

trans-1-(2-furylcarbonyl)-3,5′,8′-trifluorospiro[piperidine-4,2′(1′H)-quinazoline]-4′-amine;

cis-1-(2-thienylcarbonyl)-3,5′,8′-trifluorospiro[piperidine-4,2′(1′H)-quinazoline]-4′-amine;

trans-1-(2-thienylcarbonyl)-3,5′,8′-trifluorospiro[piperidine-4,2′(1′H)-quinazoline]-4′-amine;

(+)-(3S,2′S)-trans-1-(4-cyanobenzoyl)-3,5′,8′-trifluorospiro[piperidine-4,2′(1′H)-quinazoline]-4′-amine;

(−)-(3R,2′R)-trans-1-(4-cyanobenzoyl)-3,5′,8′-trifluorospiro[piperidine-4,2′(1H)-quinazoline]-4′-amine;

(+)-(3R,2′S)-cis-1-(4-cyanobenzoyl)-3,5′,8′-trifluorospiro[piperidine-4,2′(1′H)-quinazoline]-4′-amine;

(−)-(3S,2′R)-cis-1-(4-cyanobenzoyl)-3,5′,8′-trifluorospiro[piperidine-4,2′(1′H)-quinazoline]-4′-amine;

(3S,2′S)-trans-1-(4-cyanobenzoyl)-3,5′,8′-trifluorospiro[piperidine-4,2′(1′H)-quinazoline]-4′-amine;

(3R,2′R)-trans-1-(4-cyanobenzoyl)-3,5′,8′-trifluorospiro[piperidine-4,2′(1′H)-quinazoline]-4′-amine;

cis-1-(5-cyano-2-pyridylcarbonyl)-3,5′,8′-trifluorospiro[piperidine-4,2′(1′H)-quinazoline]-4′-amine;

(+)-(3R,2′S)-cis-1-(5-cyano-2-pyridylcarbonyl)-3,5′,8′-trifluorospiro[piperidine-4,2′(1′H)-quinazoline]-4′-amine;

(−)-(3S,2′R)-cis-1-(5-cyano-2-pyridylcarbonyl)-3,5′,8′-trifluorospiro[piperidine-4,2′(1′H)-quinazoline]-4′-amine;

trans-1-(5-cyano-2-pyridylcarbonyl)-3,5′,8′-trifluorospiro[piperidine-4,2′(1′H)-quinazoline]-4′-amine;

and acid addition salts thereof.

In one aspect the invention ineludes compounds of formula (ID)

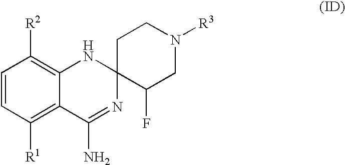

in which:

R¹ represents H, F or Cl;

R² represents H, F or CH₃;

R³ is selected from the group consisting of:

a) H; or

b) —CO—X wherein X represents:

i) a C6 to C10 aromatic ring, optionally substituted by one or more substituents selected independently from CN, Cl, F, Br, I, CF₃, OCF₃, C₁-C₃ alkyl and C₁-C₃ alkoxy;

ii) a heteroaromatic ring having from 5 to 10 ring, atoms where at least one ring atom is a heteroatom selected from O, N or S; and wherein said ring is optionally substituted bv one or more substituents selected independently from CN, Cl, F, Br, I, CF₃, OCF₃, C₁-C₃ alkyl and C₁-C₃ alkoxy or

iii) —O—(CH₂)ₙ-phenyl, wherein n represents an integer 0 to 3.

Unless otherwise indicated, the term “C1 to 3 alkyl” referred to herein denotes a straight or branched chain alkyl group having from 1 to 3 carbon atoms or a cyclic alkyl group having 3 carbon atoms. Examples of such groups include methyl, ethyl, n-propyl, i-propyl and cyclopropyl.

Unless otherwise indicated, the term “C1 to 6 alkoxy” referred to herein denotes an oxygen substituent bonded to a straight or branched chain alkyl group having, from 1 to 6 carbon atoms and/or a cyclic alkyl group having from 3 to 6 carbon atoms. Examples of such groups include methoxy, ethoxy, n-propoxy, i-propoxy, n-butoxy, i-butoxy, s-butoxy, t-butoxy, cyclopropyloxy, cyclopropylmethoxy, cyclopentyloxy, methyl cyclopentyloxy, cyclopentylmethoxy and cyclohexyloxy,

The term “C1 to 3 alkoxy” is to be interpreted analogously,

Examples of a “C6 to C10 aromatic ring” include phenyl, naphthyl, dihydronaphthyl, tetrabydronaphthyl, indanyl and indenyl.

Examples of a “heteroaromatic ring having from 5 to 10 ring atoms where at least one ring atom is a heteroatom selected from O, N or S” include furan, pyrrole, thiophene, oxazole, thiazole, imidazole, pyridine, pyrazine, pyrimidine, quinoline and isoquinoline.

The present invention ineludes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, trifluoroacetic, succinic, fumaric, maleic, methanesulphonic and benzenesulphonic acids.

According to the invention, we further provide a process for the preparation of compounds of formula (I), and opticalisomers and racemates thereof and pharmaceutically acceptable salts thereof, which comprises preparing a compound of formula (I) by:

(a) reacting a corresponding compound of formula (II) or a salt thereof

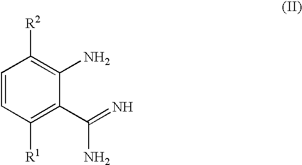

wherein R¹ and R² are as defined above,

with a compound of formula (III) or a salt thereof

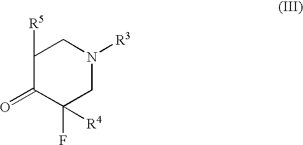

wherein R³, R⁴ and R⁵ are as defined above; or

(b) reacting a corresponding compound of formula (II) or a salt thereof,

with a compound of formula (IV) or a salt thereof

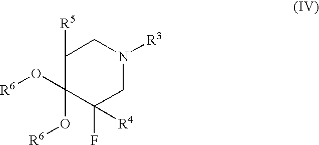

wherein R³, R⁴ and R⁵ are as defined above and R represents C₁-C₃ alkyl; or

(c) reacting a corresponding compound of formula (V) or a salt thereof,

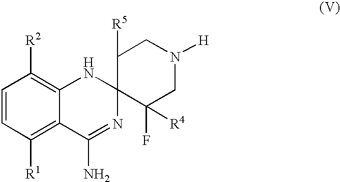

wherein R¹, R², R⁴ and R⁵ are as defined above:

with a compound of formula L—CO—X wherein X is as defined above and L represents a leaving group such as Cl or OH;

and where desired or necessary converting the resultant compound of formula (I), or another salt thereof, into a pharmaceutically acceptable salt thereof: or converting the resultant compound of formula (I) into a further compound of formula (I); and where desired converting the resultant compound of formula (I) into an optical isomer thereof.

In processes (a) and (b), the reaction will take place on stirring, a mixture of the reactants in a suitable solvent, for example a lower alkanol such as ethanol, 2-propanol or tert-butanol, at a temperature between room temperature and the reflux temperature of the solvent. The reaction may optionally be carried out under an atmosphere of an inert gas such as nitrogen or argon. The reaction time will depend inter alia on the solvent and on the reaction temperature, and may be up to 48 hours. Typically, the reaction is monitored by TLC or HPLC and is continued until the reaction is complete. In a preferred embodiment, the solvent is 2-propanol and the reaction is carried out at reflux temperature.

In process (c), the reaction is performed by reacting a compound of formula (V) with a compound of formula L—CO—X in a suitable inert solvent. Suitable leaving groups, L, include hydroxy and halides, particularly chloride. The reaction is generally carried out in the presence of a base. Potential basic additives are metal carbonate, especially alkali metal carbonates, metal oxides and hydroxides, and tertiary amine bases such as triethylamine and dilsopropylethylamine. Suitable organic solvents are those such as acetonitrile dioxane, N,N-dimethylfornamide and dichloromethane.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vacuo or by freeze drying. Suitable solvents include, for example, water, dioxan, ethanol, 2-propanol, tetrahydrofuran or diethyl ether, or mixtures thereof. The reaction may be a metathetical process or it may be carried out on an ion exchange resin.

Certain novelintermediates of formulae (III) and (IV) that are useful in the preparation of compounds of formula (I) form another aspect of the invention.

Thus, we also claim novel compounds of formula (III)

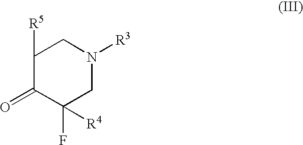

wherein R¹, R⁴ and R⁵ are as defined above, with the proviso that the compound wherein R⁴ and R⁵ each represent H and R³ represents —CO—O-tert-butyl is disclaimed.

Novel compounds of formula (IV)

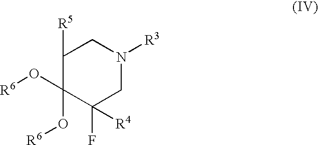

wherein R³, R⁴ and R⁵ are as defined above and R represents C₁-C₃ alkyl are also claimed.

Compounds of formula (II) may be prepared by methods that are disclosed in WO 97/14686.

In general, compounds of formula (III) may be prepared by reaction of the corresponding non-fluorinated piperidinone with a selective fluorinating agent such as Selectfluor™ [(1-(chloromethyl)-4-fluoro-1,4-diazoniabicyclo[2.2.2]octane bis(tetrafluoroborate)]. Compounds of formula (IV) may be prepared by acetalisation of corresponding compounds of formula (III), or prepared directly by reacting the corresponding non-fluorinated piperidinone with a selective fluorinating agent such as Selectfluor™ with in situ acetalisation.

Typical processes that may be used to prepare compounds of formulae (III), (IV) and (V) are illustrated in Schemes 1 to 3. The man skilled in the art will readily appreciate how such routes may be adapted to facilitate the synthesis of the exact intermediate required in order to allow the preparation of any particular compound of formula (1):

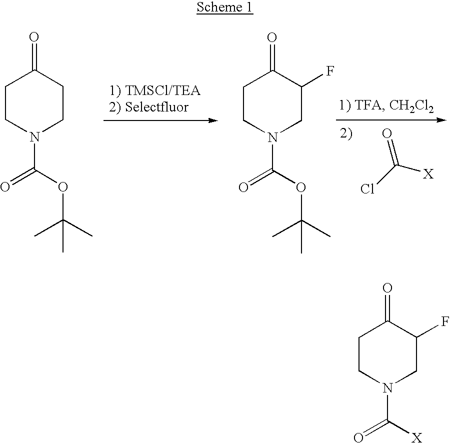

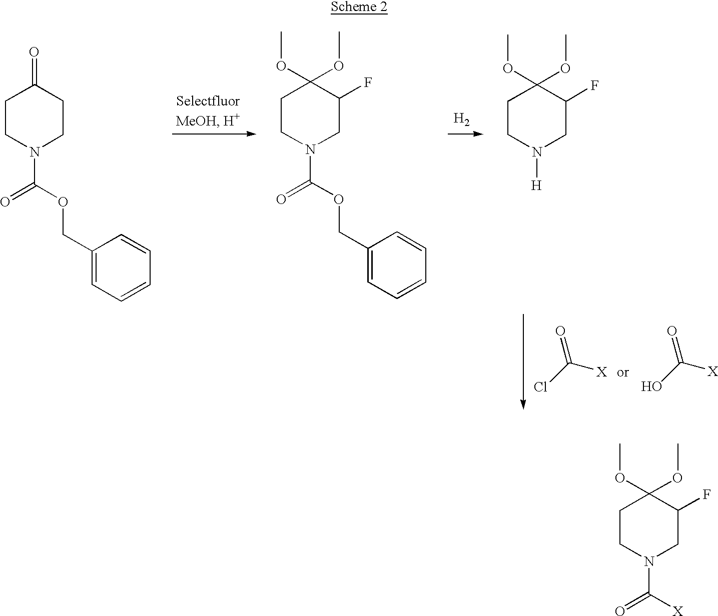

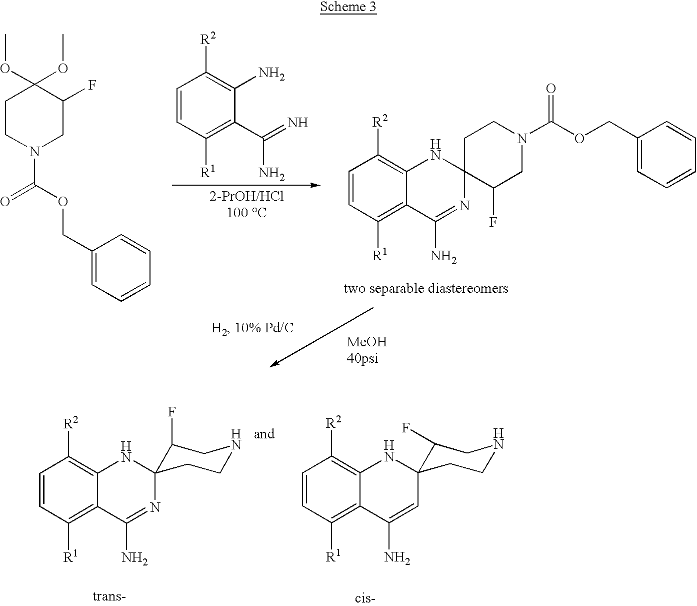

Compounds of formula L—CO—X are either known or may be prepared by known methods. As a further aspect of the invention we disclose an improved process for the preparation of such compounds wherein L represents OH and X represents cyanopyridine (Scheme 4)

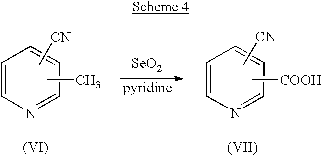

Thus, the corresponding methyl substituted cyanopyridine [formula (VI)] is oxidised by heating with selenium dioxide in pyridine, generally at about 100 ° C. The carboxylic acid derivative [formula (VII)] is then obtained directly in a single step and in excellent overall yield.

Intermediate compounds may be prepared as such or in protected form. In particular amine and hydroxy groups may be protected. Suitable protecting groups are described in the standard text “Protective Groups in Organic Synthesis”, 2nd Edition (1991) by Greene and Wuts. Amine protecting groups which may be mentioned include alkyloxycarbonyl such as t-butyloxycarbonyl, phenylalkyloxycarbonyl such as benzyloxycarbonyl, or trifluoroacetate. Deprotection will normally take place on treatment with aqueous base or aqueous acid, or hydrogenolysis.

The compounds of the invention and intermediates may be isolated from their reaction to mixtures, and if necessary further purified, by using standard techniques.

The compounds of formula (I) may exist in tautomeric, enantiomeric or diastereoisomeric forms, all of which are included within the scope of the invention. The various optical isomers may be isolated by separation of a racemic mixture of the compounds using conventional techniques, for example, fractional crystallisation or HPLC. Altematively, the individual enantiomers may be made by reaction of the appropriate optically active starting, materials under reaction conditions that will not cause racemisation.

Intermediate compounds may also exist in enantiomeric forms and may be used as purified enantiomers, diastereomers, racemates or mixtures.

The compounds of formula (I), and their pharmaceutically acceptable salts, enantiomers, racemates and tautomers, are useful because they possess pharmacological activity in animals. In particular, the compounds are active as inhibitors of the enzyme nitric oxide synthase and as such are predicted to be useful in therapy.

The compounds and their pharmaceutically acceptable salts, enantiomers, racemates and tautomers are indicated for use in the treatment or prophylaxis of diseases or conditions in which synthesis or oversynthesis of nitric oxide synthase forms a contributory part.

Among the specific conditions that may be treated are pain (including chronic pain; neuropathic pain; acute pain; caneer pain; visceral pain; pain caused by rheumatoid arthritis, migraine, etc.; pain caused by neurological complications associated with diseases such as AIDS and Alzheimer's disease and other neurodegenerative diseases); arthritic conditions (including osteoarthritis, rheumatoid arthritis, gouty arthritis); inflamed joints; rheumatoid spondylitis; inflammatory skin conditions (including eczema, psoriasis, dermatitis and sunburn); inflammatory eve conditions (e.g., uveitis (glaucoma and conjunetivitis); lung disorders in which inflammation is involved (e.g., asthma, bronchitis, pigeon fancier's disease, farmer's lung disease, chronic obstructive pulmonary disease and acute respiratory distress syndrome): bacteraemia; endotoxaemia (septic shock); and panereatitis. The compounds of the invention are also useful for the treatment of conditions of the gastrointestinal tract including: aphthous ulcers; gingivitis; Crohn's disease; atrophic gastritis; gastritis varialoforme; ulcerative colitis; coeliac disease; regionalileitis; peptic ulceration; irritable bowel syndrome; reflux oesophagiris; and damage to the gastrointestinal tract resulting from infections (e.g. by Helicobacter pylori) or due to treatment with non-steroidal anti-inflammatory drugs.

The compounds of formulae (I) and their pharmaceutically acceptable salts, enantiomers and racernates may also be useful in the treatment or prophylaxis of diseases or conditions in addition to those mentioned above. For example, the compounds may be useful in the treatment of atherosclerosis, cystic fibrosis, hypotension associated with septic and/or toxic shock, in the treatment of dysfunction of the immune system, as an adjuvant to short-tern immunosuppression in organ transplant therapy, in the control of onset of diabetes, in the maintenanee of panereatic funetion in diabetes, in the treatment of vascular complications associated with diabetes and in co-therapy with cytokines, for example TNF or interleukins.

The compounds of formulae (I) may also be useful in the treatment of hypoxia, for example in cases of cardiac arrest and stroke, neurodegenerative disorders including nerve degeneration and/or nerve necrosis in disorders such as ischaemia, hypoxia, hypoglycaemia, epilepsy, and in external wounds (such as spinal cord and head injury), hyperbaric oxygen convulsions and toxicity, dementia, for example pre-senile dementia, Alzheimer's disease and AIDS-related dementia, Sydenham's chorea, Parkinson's disease, Tourette's Syndrome, Huntington's disease, Amyotrophic Lateral Sclerosis, Multiple Sclerosis, Korsakoff's disease, imbecility relating to a cerebral vessel disorder, sleeping disorders, schizophrenia, autism, seasonal affective disorder, jet-lag and septic shock. Compounds of formulae (I) may also be expected to show activity in the prevention and reversal of drug addiction or toleranee such as tolerance to opiates and diazepines, treatment of migraine and other vascular headaches, neurogenic inflammation, in the treatment of gastrointestinal motility disorders, caneer and in the induction of labour.

The compounds of formula (I) are particularly useful in the treatment and alleviation of acute or persistent inflammatory or neuropathic pain, or pain of central origin.

For the treatment of pain associated with migraine, the compounds of formula (I) are expected to be particularly useful either alone, or in combination with other assents. particularly in combination with a 5HT_{1B/1D} (serotonin-1B/1D) agonist. Thus, the compounds of formula (I), and pharmaceutically acceptable derivatives thereof, may also be advantageously used in combination with a 5HT_{1B/1D} (serotonin-1B/1D) agonist or a pharmaceutically acceptable derivative thereof. Particularly preferred 5HT_{1B/1D} (serotonin-1B/1D) agonists include sumatriptan, naratriptan, rizatriptan, zolmitriptan, almotriptan, eletriptan and frovatriptan. Zolmitriptan is especially preferred. The NOS inhibitor and the 5HT_{1B/1D} (serotonin-1B/1D) agonist may either be formulated together within the same pharmaceutical composition for administration in a single dosage unit, or each component may be individually formulated such that separate dosa(ges may be administered either simultaneously or sequentially,

Prophylaxis is expected to be particularly relevant to the treatment of persons who have suffered a previous episode of, or are otherwise considered to be at inereased risk of, the disease or condition in question. Persons at risk of developing a particular disease or condition generally include those having a family history of the disease or condition, or those who have been identified by genetic testing or screening to be particularly susceptible to developing the disease or condition.

Thus according to a further aspect of the invention we provide a compound of formula (I), or an optical isomer or racemate thereof or a pharmaceutically acceptable salt thereof, for use as a medicament.

According to another feature of the invention we provide the use of a compound of formula (I) or an optical isomer or racemate thereof or a pharmaceutically acceptable salt thereof, in the manufacture of a medicament for the treatment or prophyylaxis of the aforementioned diseases or conditions: and a method of treatment or prophylaxis of one of the aforementioned diseases or conditions which comprises administering a therapeutically effective amount of a compound of formula (I), or an optical isomer or racemate thereof or a pharmaceutically acceptable salt thereof, to a person suffering from or susceptible to such a disease or condition.

The compounds of the present invention may also be used advantageously in combination with a second pharmaceutically active substance, particularly in combination with a selective inhibitor of the inducible isoform of cyclooxygenase (COX-2). Thus, in a further aspect of the invention there is provided the use of a compound of formula (I) or a pharmaceutically acceptable salt, enantiomer or racemate thereof, in combination with a COX-2 inhibitor for the treatment of pain and inflammatory disease. And there is also provided a method of treating, or reducing the risk of, pain and inflammatory disease in a person suffering from or at risk of, said disease or condition, wherein the method comprises administering to the person a therapeutically effective amount of a compound of formula (I) or a pharmaceutically acceptable salt, enantiomer or racemate thereof in combination with a COX-2 inhibitor.

The compounds of formula (I), and optical isomers and racemates thereof and pharmaceutically acceptable salts thereof, may be used on their own, or, preferably, in the form of appropriate medicinal formulations (pharmaceutical compositions). Conventional procedures for the selection and preparation of suitable pharmaceutical formulations are described in, for example, “Pharmaceuticals—The Science of Dosage Form Designs”, M. E. Aulton, Churchill Livingstone, 1988.

For the above mentioned therapeutic indications, the dosage administered will, of course, vary with the compound employed, the mode of administration and the treatment desired. However, in general, satisfactory results are obtained when the compounds are administered to a human at a daily dosage of between 0.5 mg and 2000 mg (measured as the active ingredient) per day, particularly at a daily dosage of between 2 mg and 500 mg.

The compounds of formula (I) may be incorporated into pharmaceutical compositions and used in the treatment of any of the diseases associated with excessive levels of nitric oxide. Among the conditions amenable to treatment are pain (including pain due to migraine). inflammatory conditions (e.g., rheumatoid arthritis, osteoarthritis, and inflammatory bowel disease) and autoimmune disease (e.g. multiple sclerosis). The total daily dosage of compound administered to a patient should be at least the amount required to reduce or eliminate one or more symptoms associated with the condition being treated. For example, in the treatment of pain, sufficient agent should be administered to reduce or eliminate the discomfort experienced by a patient. The actual dose selected for an individual patient will be determined by the attending physician based upon clinical conditions and using methods well known in the art. Agents may be provided in either a single or multiple dosage regimen, that is, a patient may be administered compounds one or more times a day.

Any route of administration and dosage form is compatible with the invention, and a therapeutic agent may be administered as either the sole active ingredient or in combination with other therapeutically active drugs. For example, the compounds may be administered to patients in combination with other agents used for the clinical management of pain, for example together with opiates such as morphine. Routes of delivery compatible with the invention include parenteral, peroral, internal, pulmonary, rectal, nasal, vaginal, lingual, transdermal, topical, intravenous, intraarterial, intramuscular, intraperitoneal, intracutaneous, and subcutaneous routes. Specific dosage forms that may be used include tablets, pills, capsules, powders, aerosols, suppositories, skin patches, parenterals, and oral liquids, including oil aqueous suspensions, solutions, and emulsions. Sustained release dosage forms may also be used.

Therapeutic agents may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Colouring and flavouring agents may also be added to preparations designed for oral administration. Solutions can be prepared using water or physiologically compatible organic solvents such as ethanol, 1,2-propylene glycol, polyglycols, dimethyl sulfoxide, fatty alcohols, triglycerides, partial esters of glycerin, and the like. Parenteral compositions may be prepared using conventional techniques and include sterile isotonic saline, water, 1,3-butane diol, ethanol, 1,2-propylene glycol, polyglycols mixed with water, Ringer's solution, etc.

If desired, a patient may be initially given a relatively low dose of therapeutic agent in order to determine whether any adverse side effects are experienced. This may be particularly important in cases where a patient is taking other medications or has clinical characteristics that suggest that they may not be able to tolerate high drug dosages. If adverse side effects are not experienced by the patient, dosage may be gradually increased until a satisfactory alleviation of symptoms is achieved. For example, the dosage given to a patient suffering from chronic arthritic pain may be gradually increased until the patient experiences appropriate relief.

Compounds of formula (I) are particularly advantageous in that they possess high potency for inhibition of the iNOS isoform and also exhibit a high degree of selectivity for inhibition of the iNOS isoform (compared to inhibition of eNOS and bNOS). Compounds of formula (I) also have markedly different physicochemical properties when compared to the compounds disclosed in WO 97/14686. For example, in general they exhibit improved oral bioavailability, and are thereby more suited to use as pharmaceutical agents.

The invention is illustrated but in no way limited by the following examples:

### Intermediate 1

### tert-Butyl 3-fluoro-4-oxo-1-piperidinecarboxylate

The title compound was prepared from tert-butyl 4-oxo-1-piperidine carboxylate according, to a literature procedure (Niel et al, J. Med. Chem., 2087 (1999)).

### Intermediate 2

### Benzyl 3-fluoro-4,4-dimethoxy-1-piperidinecarboxylate

A mixture of commercially available benzyl 4-oxo-1-piperidinecarboxylate (10 g, 42.9 mmol), Selectfluor™ (25 g, 70.6 mmol) and concentrated sulfuric acid (3 mL) in methanol (150 mL) was heated at 50° C. under a nitrogen atmosphere for 18 h. Water (300 mL) was added and the resultin, mixture was extracted with ethyl acetate (3×300 mL). The combined organic phases were washed with brine (300 mL) and dried over sodium sulfate. Concentration and flash chromatography (ethyl acetate: heptane (50:50) on silica gel 60 gave the title compound (9.8 g, 76%) as a colorless thick oil.

¹H NMR (CDCl₃): δ1.85 (2H, m), 2.8-3.0 (1H, m), 3.1-3.3 (1H, m), 3.2 (3H, s), 3.3 (3H, s),3.95-4.15 (1H, m), 4.3-4.7 (2H, m), 5.15 (2H, bs), 7.3 (5H, m).

MS ^{m}/z: 298 (M+1).

### Intermediate 3

### 3-Fluoro-4,4-dimethoxypiperidine

A mixture of benzyl 3-fluoro-4,4-dimethoxy-1-piperidinecarboxylate (1.03 g, 3.45 mmol) and 10% Pd/C (0.34 mmol) in methanol (75 mL) was shaken under an atmosphere of hydrogen at 45 psi for 18 h. Filtration and concentration gave the title compound (568 mg, 100%).

1H NMR (free amine, CDCl₃): δ1.64-1.73 (1H, m),1.86 (1H, br d. J 1.5 Hz), 2.65 (1H, br t, J 13.2 Hz), 2.90-3.04 (2H, m), 3.18-3.29 (1H, m), 3.22 (3H, s), 3.29 (3H, s):

MS ^{m}/z: 164 (M+H).

### Intermediates 4

All 2-amino-benzamidine derivatives were prepared according to WO 97/14686.

### Intermediates 5

### General Procedure A for the Synthesis of N-acylated-3-fluoro-4-piperidinones

Trifluoroacetic acid was added to a suspension of tert-butyl 3-fluoro-4-oxo-1-piperidinecarboxylate in dry dichloromcthane. The reaction mixture was stirred under a nitrogen atmosphere at room temperature for 30 minutes. The mixture was concentrated under reduced pressure and the resulting oil was dissolved in dry tetrahydrofuran and cooled to 0° C. The acyl chloride (1.2 eq.) was then added dropwise followed by triethylamine (1.4 eq.). The reaction was allowed to warm to room temperature and was then stirred under a nitrogen atmosphere for 18 h. The reaction was quenched by the addition of water and the resulting layers were separated. The aqueous layer was extracted with ethyl acetate (3×), and the combined organic layers were washed with saturated aqueous sodium hydrogen carbonate (1×) and saturated aqueous sodium chloride (1×), then dried over anhydrous sodium sulphate, filtered and concentrated to give the desired product, which was further purified by MPLC.

Using this general procedure, the following compounds were prepared:

a) 1-(4-Cyanobenzoyl)-3-fluoro-4-piperidinone

tert-Butyl 3-fluoro-4-oxo-1-piperidinecarboxylate (1.01 g, 4.65 mmol) and trifluoroacetic acid (6 mL) in dichloromethane (12 mL) gave the deprotected intermediate. This material in tetrahydrofuran (15 mL) was treated with 4-cyanobenzoyl chloride (920 mg. 5.58 mmol) and triethylamine (0.91 mL, 6.51 mmol). MPLC (silica gel 60, 50 to 100% ethyl acetate in heptane) then gave the title compound (646 mg, 57%).

¹H NMR (CDCl₃): δ1.21-2.00 (2H, m), 3.08-4.21 (2H, m), 4.40-5.18 (1H, m), 7.6 (2H, d, J=8.1 Hz), 7.78 (1H, d, J=8.1 Hz);

MS ^{m}/z: 266 (M+NH₄).

b) 1-(4-Chlorobenzoyl)-3-fluoro-4-piperidinone

tert-Butyl 3-fluoro-4-oxo-1-piperidinecarboxylate (0.64 g, 2.95 mmol) in dichloromethane (8 mL) and trifluoroacetic acid (4 mL) gave an intermediate which was dissolved in tetrahydrofuran (10 mL). 4-Chlorobenzoyl chloride (0.45 mL, 0.60 g, 3.52 mmol) and Et₃N (0.58 mL, 0.42 g, 4.16 mmol) were added. The product was purified by MPLC (silica gel 60, hexane: ethyl acetate, 1:1) to give the title compound (0.62 g, 82%) as a white solid.

¹H-NMR (CDCl₃): δ1.8-3.0 (3 H, m), 3.4-4.2 (4H, m), 7.6 (2H, m), 7.8 (2H, m);

MS ^{m}/z: 256 (M⁺).

c) 3-Fluoro-1-(2-thienylcarbonyl)-4-piperidinone

Yield (43%).

¹H-NMR (CDCl₃): δ2.7 (2H, m), 3.6 (2H, m), 4.4-5.0 (3H, m), 7.12 (1H, dd, J=4.4, 3.7 Hz),7.43 (1H, d, J=3.7 Hz), 7.55 (1H, d, J=4.4 Hz).

### Intermediates 6

### General procedure B for the synthesis of N-acylated-3-fluoro-piperidinone dimethyl acetals using carboxylic acids

O-(7-Azabenzotriazol-1-yl)-N,N,N′,N′-tetramethyluronium hexafluorophosphate (HATU, 1.2 eq.) was added to a solution of the carboxylic acid (1 eq.) in dry N,N-dimethylformamide at 0° C. 3-Fluoro-4,4-dimethoxypiperidine (1.2 eq.) was then added followed by N,N-diisopropylethylamine (3.0 eq.). The reaction was allowed to warm to room temperature and was then stirred under a nitrogen atmosphere for 18 h. The reaction was quenched with saturated aqueous ammonium chloride and the layers separated. The aqueous layer was extracted with ethyl acetate (3×) and the combined organic layers were washed with 10% aqueous hydrochloric acid (1×), water (1×) and saturated aqueous sodium chloride (1×), then dried over anhydrous sodium sulphate, filtered and concentrated, to give the desired product which was purified by MPLC. Using this general procedure, the following compound was prepared:

### 1-(6-Cyano-3-pyridylcarbonyl)-4,4-dimethoxy-3-fluoropiperidine

6-Cyano-3-pyridylcarboxylic acid (424 mg, 2.86 mmol) in N,N-dimethyltomlamide (30 mL) with HATU (1.30 g, 3.43 mmol), Hunig's base (1.49 mL, 8.57 mmol) and 3-fluoro-4,4-dimethoxypiperidine (560 mg, 3.43 mmol). MPLC (silica gel 60, 30 to 100% ethyl acetate in heptane) gave the title compound (658 mg, 66%).

¹H NMR (CDCl₃): δ1.83-1.97 (1H, m), 2.00-2.08 (1H, m), 3.27 (3H, s), 3.29 (3H, s), 3.21-3.33 (1H, m) 3.39-3.56 (1H, m), 3.76 (1H, br t, J 11.4 Hz), 4,49 (1H, d, J 48.3 Hz), 4.64 (1H, br d, J 10.2 Hz), 7.76 (1H, d, J 8.1 Hz), 7.90 (1H, dd, J 5.9. 2.2 Hz), 8.75 (s. 1H);

MS ^{m}/z: 293 (M⁺).

### Intermediates 7

### Alternative general procedure C for the synthesis of N-acylated-3-fluoro-piperidinone dimethyl acetals using carboxylic acids

To a solution of the carboxylic acid (1 eq.) in N,N-dimethylformamide (5 mL) was added carbonyldiimidazole (1.2 eq.) and the resulting mixture was stirred for 30 minutes at room temperature. A solution of 3-fluoro-4,4-dimethoxypiperidine (1 eq.) in N,N-dimethylformamide (4 mL) was added. The reaction mixture was stirred for 8 h at room temperature, diluted with water (100 mL) and extracted with ethyl acetate (4×50 mL). The combined organic phases were washed with brine and dried using, sodium sulphate. After concentration, the residue was purified by MPLC.

### Intermediates 8

### General procedure D for the synthesis of N-acylated-3-fluoro-4-piperidinone dimethyl acetals using a acid chlorides

3-Fluoro-4,4-dimethoxypiperidine was dissolved in dry tetrahydrofuran and cooled to 0° C. The acid chloride (1.2 eq) was added followed by the dropwise addition of triethylamine (1.4 eq). The reaction was allowed to warm to room temperature and stirred under a nitrogen atmosphere for 4 h. The reaction was quenched with water and the resulting layers were separated. The aqueous layer was extracted with ethyl acetate (3×) and the combined organic layers were washed with saturated aqueous sodium hydrogen carbonate (1×) and saturated aqueous sodium chloride (1×), then dried over anhydrous sodium sulphate, filtered and concentrated to give the desired product which was purified by MPLC. Using, this general procedure, the following, compounds were prepared:

a) 1-(4-Chlorobenzoyl)-4,4-dimethoxy-3-fluoropiperidine

White solid. Yield (83%); ¹H-NMR (CDCl₃): δ1.9-2.0 (2H, m), 2.8 (1H, broad), 3.26 (3H, s), 3.29 (3H, s), 3.9 (1H, broad), 4,4 (1H, broad), 4.5-4.6 (2H, m), 7.4 (4H, m);

MS^{m}/z: 302 (M+H).

b) 4,4-Dimethoxy-3-fluoro-1-(4-methylbenzoyl)piperidine

From 3-fluoro-4,4-dimethoxypiperidine (360 mg, 2.20 mmol) in tetrahydrofuran (5 mL) with p-toluoyl chloride (409 mg, 2.65 mmol) and triethylamine (0.43 mL. 3.09 mmol). MPLC (silica gel 60, 50 to 100% ethyl acetate in heptane) gave the title compound (528 mg, 85%).

¹H NMR (CDCl₃): δ1.78-2.08 (2H, m), 2.76-3.15 (2H, m), 3.25 (3H, s), 3.29 (3H, s) 3.94-4.16 (1H, m), 4.32-4.96 (2H, m), 7.20 (2H, d, J=8.1 Hz), 7.32 (2H, d, J=8.1 Hz);

MS ^{m}/z: 282 (M+H).

c) 4,4-Dimethoxy-3-fluoro-1-(2-thienylcarbonyl)piperidine

From 3-fluoro-4,4-dimethoxypiperidine (347 mg, 2.12 mmol) in tetrahydrofuran (4 mL) with 2-thiophene carbonyl chloride (374 mg, 2.55 mmol) and triethylamine (0.41 mL, 2.97 mmol). MPLC (silica gel 60, 50 to 100% ethyl acetate in heptane) gave the title compound (505 mg, 87%) as a yellow oil,

¹H NMR (CDCl₃): δ1.83-2.08 (2H, m), 2.97 (1H, br s), 3.27 (3H, s), 3.31 (3H, s), 3.40-3.51 (1H, m), 4,25-4.71 (3H, br m), 7.04 (1H, t, J 4.0 Hz), 7.32 (1H, d, J 2.9 Hz), 7.44 (1H, d, J 5.1 Hz);

MS ^{m}/z: 274 (M+H).

d) 4,4-Dimethoxy-3-fluoro-1-(4-methylbenzoyl)piperidine

Colourless syrup. Yield (79%).

¹H-NMR (CDCl₃): δ1.9 (2H, broad), 2.37 (3H, s), 2.8 (1H, broad), 3.25 (3H, S) 3.29 (3H, s), 3.7 (1H, broad), 4.0 (1H, broad) 4.4-4.5 (2H, m), 7.20 (1H, d, J 7.3 Hz), 7.32 (2H, d, J 8.1 Hz);

MS ^{m}/z: 282 (M+H).

e) 4,4-Dimethoxy-3-fluoro-1-(2-furylcarbonyl)piperidine

Colourless syrup. Yield (73%).

¹H-NMR (CDCl₃): δ1.9 (2H, m) 3.0 (1H, broad), 3.27 (3H, S), 3.31 (3H, s), 3.5 (1H, broad), 4.6-4.8 (3H, m). 6.48 (1H dd, J 3.7, 2.2 Hz), 702 (1H, d, J 3.7 Hz), 7.49 (1H, d, J 2.2 Hz);

MS ^{m}/z: 258 (M+H).

### EXAMPLES

### General procedures E and F for the synthesis of fluoro-piperidine spirocycles

General Procedure E

A mixture of the N-acylated-3-fluoropiperidin-4-one or the N-acylated-3-fluoropiperidin-4-one dimethyl acetal (1.1 eq.) and the 2-amino-benzamidine hydrochloride salt (1 eq.) in 2-propanol was heated at reflux for 4 to 24 h. The reaction was monitored by TLC or HPLC. Upon evaporation of the solvent, ethyl acetate (20 mL) and triethylamine (0.37 mL) were added. The suspension was stirred for 30 minutes at room temperature, washed with water (5 mL) and the organic layer was then separated and dried using sodium sulphate. After concentration, the residue was purified by MPLC (silica gel 60, methanol dichloromethane : aqueous ammonia (1:10:0.01) to give the fluoro-piperidine spirocycle as two separated diastereorners. In certain cases, each diastereomer was then subjected to chiral HPLC to give two enantiomers.

General Procedure F

A mixture of the N-acylated-3-fluoropiperidin-4-one or the N-acylated-3-fluoropiperidin-4-one dimethyl acetal (1.1 eq.) and the 2-amino-benzamidine hydrochloride salt (1 eq.) in 2-propanol was heated at reflux for 4 to 24 h. The reaction was monitored by TLC or HPLC. After concentration, the residue was purified by MPLC (silica gel 60, 0 to 10% methanol in dichloromethane containing 0.1% aqueous ammonia) to give the fluoro-piperidine spirocycle as two separated diastereomers. In certain cases, each diastereomer was then subjected to chiral HPLC to give two enantiomers.

### Examples 1 and 2

### Cis- and Trans-Diastereomers of 1-(4-Cyanobenzoyl)-3-fluorospiro[piperidine-4,2′(1′H)-quinazoline]-4′-amine trifluoroacetate

Using General Procedure F, 1-(4-cyanobenzoyl)-3-fluoro-4-piperidinone (360 mg. 1.46 mmol) and 2-aminobenzamidine dihydrochloride (209 mg. 1.22 mmol) in 2-propanol (8 mL) gave the title diastereomers.

Cis-isomer (136 mg, 31%). ¹H NMR (free amine, CD₃OD): δ1.93-2.35 (2H, m), 3.73 (1H, br t, J 13.9 Hz), 3.46-3.71 (2H, m), 3.75-3.87 (1H, m), 4.50-4.77 (1H, m), 6.90 (1H, t, J 7.7 Hz), 7.00 (1H, d, J 8.1 Hz), 7.48-7.53 (1H, m), 7.57 (2H, br d, J 8.1 Hz), 7.73-7.77 (1H, m), 7.80-7.88 (2H, m);

MS ^{m}/z: 364 (M+H).

Tians-isomer (103 mg, 23%). ¹H NMR (free amine, CD₃OD): δ1.70-2.09 (2H, m), 3.28-3.73 (4H, m), 4.30-4.78 (1H, m), 6.65-6.77 (2H, m), 7.23 (1H, t, J 7.0 Hz), 7.42 (1h, t, J 8.4 Hz), 7.55 (2H, t, J 7.3 Hz), 7.75-7.86 (2H, m);

MS ^{m}/z: 364 (M+H).

### Examples 3 and 4

### Cis-and Trans-Diastereomers of 1-(4-Chlorobenzoyl)-3-fluorospiro[piperidine-4,2′(1′H)-quinazoline]-4′-amine trifluoroacetate

Using General Procedure F, 1-(4-chlorobenzoyl)-4,4-dimethoxy-3-fluoropiperidine (272 mg, 0.90 mmol) and 2-aminobenzamidine dihydrochloride (129 mg, 0.75 mmol) in 2-propanol (5 mL) gave the title diastereomners.

Cis-isomer (110 mg, 39%). ¹H NMR (tree amine, CD₃OD): δ2.00-2.39 (2H, m), 3.38-3.99 (4H, m), 4.45-4.79 (1H, m), 6.91 (1H, t, J 7.3 Hz), 7.06 (1H, d, J 8.1 Hz), 7.40-7.54 (5H, m), 7.78 (1H, d, J 7.3 Hz);

MS ^{m}/z: 373 (M+H).

Trans-isomer (54 mg, 19%). ¹H NMR (trifluoroacetate salt, DMSO-d₆): δ2.0 (2H, m), 3.2-3.8 (3H, m), 4.1-4.40 (1H, m), 4.6-4.8 (1H, m), 6.8 (1H, m), 6.90 (1H, m), 7.4 (2H, m), 7.5 (3H, m), 7.8 (2H, m), 8.4 (1H, m), 9.3 (1H, m), 9.9 (1H, m);

MS ^{m}z: 373 (M+H).

### Examples 5 and 6

### Cis-and Trans-Diastereomers of 1-(6-Cyano-3-pyridylcarbonyl)-3-fluorospiro[piperidine-4.2′(1′H)-quinazoline]-4′-amine trifluoroacetate

Using General Procedure F, 1-(6-cyano-3-pyridylcarbonyl)-4,4-dimethoxy-3-fluoropiperidine (186 mg, 0.63 mmol) and 2-aminobenzamidine dihydrochloride (91 mg, 0.53 mmol) in 2-propanol (8 mL) gave the title diastereomers.

Cis-isomer (45 mg, 23%). ¹H NMR (free amine, CD₃OD): δ1.95-2.38 (2H, m), 3.29-3.89 (4H, m), 4.56-4.74 (1H, m), 6.90 (1H, t, J 7.3 Hz), 7.03 (1H, d, J 8.1 Hz), 7.50 (1H, t, J 7.3 Hz), 7.75 (1H, d, J 8.1 Hz), 7.92-8.08 (2H, m), 8.72 (1H, br s);

MS ^{m}/z: 365 (M+H).

Trans-isomer (29 mg, 15%). ¹H NMR (free amine, CD₃OD): δ1.73-2.09 (2H, m), 3.25-3.74 (4H, m), 4.37-4.78 (1H, m), 6.65-6.78 (2H, m), 7.24 (1H, t, J 7.0 Hz), 7.43 (1H, t, J 8.4 Hz), 7.90-8.01 (2H, m), 8.72 (1H, d, J 13.9 Hz);

MS ^{m}/z: 365 (M+H).

### Examples 7 and 8

### Cis-and Trans-Diastereomers of 3-Fluoro-1-(4-methylbenzoyl)-spiro[piperidine-4,2′(1′H)-guinazoline]-4′-amine trifluoroacetate

Using General Procedure F, 4,4-dimethoxy-3-fluoro-1-(4-methylbenzoyl) piperidine (525 mg, 1.87 mmol) and 2-aminobenzamidine dihydrochloride (267 mg, 1.56 mmol) in 2-propanol (5 mL) gave the title diastereomers.

Cis-isomer (105 mg. 14%). ¹H NMR (trifluoroacetate salt, CD₃OD): δ1.95-2.38 (2H, m), 2.36 (3H, s), 3.29-4.09 (3H, m), 4.50-4.79 (2H, m), 6.92 (1H, t, J 7.7 Hz), 7.00 (1H, d, J 8.1 Hz), 7.23-7.34 (4H, m), 7.53 (1H, t, J 7.7 Hz), 7.77 (1H, d, J 8.1 Hz);

MS ^{m}/z: 353 (M+H);

Trans-isomer (103 mg, 14%). ¹H NMR (trifluoroacetate salt, CD₃OD): 1.95-2.39 (2H, m), 2.36 (3H, s), 3.30-4.08 (3H, m), 4.42-4.79 (2H, m), 6.88 (1H, t, J 7.3 Hz), 6.93 (1H, d, J 8.1 Hz), 7.30 (4H, br t, J 9.1 Hz), 7.51 (1H, t, J 7.3 Hz), 7.72 (1H, d, J 8.1 Hz):

MS ^{m}/z: 353 (M+H).

### Examples 9 and 10

### Cis-and Trans-Diastereomers of 3-Fluoro-1-(2-furylcarbonyl)-spiro[piperidine-4,2′(1′H)-quinazoline]-4′-amine trifluoroacetate

Using General Procedure F, 4,4-dimethoxy-3-fluoro-1-(2-furylcarbonyl) piperidine (292 mg, 1.38 mmol) and 2-aminobenzamidine dihydrochloride (198 mg, 1.15 mmol) in 2-propanol (5 mL) gave the title diastereomers.

Cis-isomer (135 mg, 36%). ¹H NMR (trifluoroacetate salt, CD₃OD): δ2.08-2.37 (2H, m), 3.33-3.89 (2H, m), 4.45-4.85 (3H, m), 6.58 (1H, s), 6.93 (1H, t, J 7.7 Hz), 7.02 (1H, d, J 8.1 Hz), 7.07 (1H, s), 7.53 (1H, t, J 7.7 Hz), 7.68 (1H, s), 7.78 (1H, d, J 8.1 Hz);

MS ^{m}/z: 329 (M+H);

Trans-isomer (103 mg, 27%). ¹H NMR (trifluoroacetate salt, CD₃OD): 1.85-2.18 (2H, m), 3.30-3.87 (2H, m), 4.34-4.67 (3H, m), 6.56 (1H, s), 6.71-6.81 (2H, m), 7.01 (1H, s), 7.31 (1H, t, J 7.7 Hz), 7.51 (1H, d, J 7.3 Hz), 7.65 (1H, s);

MS ^{m}/z: 329 (M+H).

### Examples 11 and 12

### Cis-and Trans-Diastereomers of 3-Fluoro-1-(2-thienylcarbonyl)-spiro[piperidine-4,2′(1′H)-guinazoline]-4′-amine trifluoroacetate

Using General Procedure F, 4,4-dimethoxy-3-fluoro-1-(2-thienylcarbonyl) piperidine (500 mg, 1.83 mmol) and 2-amninobenzamidine dihydrochloride (261 mg. 1.52 mmol) in 2-propanol (5 mL) gave the title diastereomers.

Cis-isomer (135 mg, 26%). ¹H NMR (trifluoroacetate salt, CD₃OD): δ2.06-2.36 (2H, m). 3.30-3.74 (2H, m), 4.39-4.87 (3H, m), 6.85-6.96 (1H, m), 6.96-7.05 (1H, m), 7.05-7.13 (1H, m), 7.36-7.43 (1H, m), 7.48-7.57 (1H, m). 7.60-7.66 (1H, m), 7.75-7.81 (1H, m):

MS ^{m}/z: 345 (M+H);

Trans-isomer (130 mg, 25%). ¹H NMR (trifluoroacetate salt. CD₃OD): 1.90-2.38 (2H, m). 3.40-3.78 (2H, m), 4.30-4.75 (3H, m), 6.86-6.98 (2H, m), 7.08-7.15 (1H, m), 7.41 (1H, d, J 2.9 Hz), 7.52 (1H, t, 7.7 Hz), 7.65 (1H, d, J 4.4 Hz), 7.74 (1H, d, J 8.1 Hz); MS ^{m}/z: 345 (M+H).

### Examples 13 and 14

### Cis-and Trans-Diastereomers of 3,5′-difluoro-1-(2-thienylcarbonyl)-spiro[piperidine-4,2′(1′H)-quinazoline]-4′-amine trifluoroacetate

Using General Procedure E, 3-fluoro-1-(2-thienylcarbonyl)-4-piperidinone (333 mg, 1.46 mmol) and 2-amino-6-fluorobenzamidine dihydrochloride (275 mg, 1.21 mmol) in 2-propanol (8 mL) gave the title diastereomers.

Cis-isomer (3.21 mg, 56%), light yellow solid. ¹H-NMR (DMSO-d₆): δ2.0 (1H, m), 2.1 (1H, m), 3.4 (2H, m), 4.3 (2H, m), 4.75 (1H, d, J 45.4 Hz), 6.66 (1H, dd, 12.4, 8.1 Hz). 6.79 (1H, d, J 8.8 Hz), 7.09 (1H, dd, J 5.1, 3.7 Hz), 7.37 (1H, d, J 3.7 Hz), 7.5 (1H, m), 7.74 (1H, d, J 5.1 Hz), 8.15 (1H, s), 8.61 (1H, d, J 4.8 Hz), 8.88 (1H, d, J 6.4 Hz), 10.22 (1H, s);

MS ^{m}/z: 363 (M+H);

Trans-isomer (170 mg, 30%), light yellow solid. ¹H-NMR (DMSO-d₆): δ2.0 (2H, m), 3.5 (2H, m), 4.2 (2H, m), 4.85 (1H d, J 47.6 Hz), 6.64 (1H, dd, J 11.7, 8.1 Hz), 6.72 (1H, d, J 8.8 Hz), 7.09 (1H, dd, J 5.1, 3.7 Hz), 7.38 (1H, d, J 3.7 Hz), 7.5 (1H, m), 7.74 (1H, d, J 5.1 Hz), 8.23 (1H, s), 8.71 (1H, s), 8.87 (1H, s), 10.03 (1H, s);

MS ^{m}/z: 363 (M+H).

### Examples 15 and 16

### Cis-and Trans-Diastereomers of 3,5′-Difluoro-1-(4-chlorobenzoyl)-spiro[piperidine-4,2′(1′H)-guinazoline]-4′-amine trifluoroacetate

Using General Procedure E, 1-(4-chlorobenzoyl)-3-fluoro-4-piperidinone (307 mg. 1.20 mmol) and 2-amino-6-fluorobenzamidine dihydrochloride (228 mg, 1.20 mmol) in 2-propanol (6 mL) gave the title diastereomers.

Cis-isomer (272 mg, 45%), light yellow solid ¹H-NMR (DMSO-d₆): δ2.0 (2H, m) 3.4 (2H, m), 3.7 (1H, broad), 4.4-4.9 (2H, m), 6.69 (1H, dd, J 11.7, 8.1 Hz), 6.81 (1H, d, J 8.1 Hz), 7.38 (2H, d, J 8.1 Hz), 7.5 (3H, m), 8.15 (1H, s), 8.62 (2H, s broad), 9.98 (1H, s);

MS ^{m}/z: 391 (M+H);

Trans-isomer (111 mg, 18%), white solid. ¹H-NMR (DMSO-d₆): δ2.0 (2H, m), 3.4 (2H, m), 3.7 (1H, broad), 4.3 (1H, broad), 4.8 (1H, m), 6.63 (1H, dd, J 11.7, 8.0 Hz), 6.72 (1H, d, J 8.1 Hz), 7.4 (2H, m), 7.5 (3H, m), 8.22 (1H, s), 8.70 (1H, s), 8.81 (1H, s), 9.98 (1H, s);

MS ^{m}/z: 391 (M+H);

### Examples 17 and 18

### Cis-and Trans-Diastereomers of 3,5′-difluoro-1-(4-cyanobenzoyl)-spiro[piperidine-4,2′(1′H)-guinazoline]-4′-amine trifluoroacetate

Using General Procedure E, 1-(4-cyanobenzoyl)-3-fluoro4-piperidinone (352 mg, 1.43 mmol) and 2-amino-6-fluorobenzamidine dihydrochloride (270 mg, 1.20 mmol) in 2-propanol (8 mL) gave the title diastereomers.

Cis-isomer (285 mg, 48%), light yellow solid. ¹H-NMR (DMSO-d₆): δ1.9-2.0 (2H, m), 3.2-3.4 (3H, m), 4.4 (1H, m), 4.63 (0.6H, d, J 46.2 Hz), 4.84 (0.4H, d, J 46.2 Hz), 6.66 (1H, dd, J 11.7, 8.1 Hz), 6.77 (1H, d, J 8.8 Hz), 7.5 (3H, m), 7.9 (2H, m), 8.11 (0.4H, s), 8.15 (0.6H, s), 8.60 (1H, s), 8.83 (1H, s), 10.12 (0.4H, s), 10.18 (0.6H, s);

MS ^{m}/z: 382 (M+H);

Trans-isomer (160 mg, 27%), white solid ¹H-NMR (DMSO-d₆): δ1.9-2.0 (2H, m), 3.4 (3H, m), 4.3 (1H, m), 4.75 (0.6H, d, J 46.1 Hz), 4.92 (0.4H, d, J 45.4 Hz), 6.6 (2H, m), 7.5 (3H, m), 7.9(2H, m), 8.21 (1H, s), 8.69 (1H, s), 9.13 (1H, s) 10.22 (0.4H, s), 10.33 (0.6H, s);

MS ^{m}/z: 382 (M+H).

### Examples 19 and 20

### Cis-and Trans-Diastereomners of 3,5′-difluoro-1-(2-furylcarbonyl)-spiro[piperidine-4,2′(1′H)-guinazoline]-4′-amine trifluoroacetate

Using General Procedure E, 4,4-dimethoxy-3-fluoro-1-(2-furylcarbonyl) piperidine (371 mg, 1.44 mmol) and 2-amino-6-fluorobenzamidine dihydrochloride (272 mg, 1.20 mmol) in 2-propanol (8 mL) gave the title diastereomers.

Cis-isomer (321 mg, 58%), light yellow solid. ¹H-NMR (DMSO-d₆): δ2.0 (2H, m), 3.5 (2H, broad), 4.4 (2H, m), 4.78 (1H, d, J 46.1 Hz), 6.61 (1H, dd, J 3.7, 1.5 Hz), 6.68 (1H, dd, J 11.7, 8.1 Hz), 6.83 (1H, d, J 8.1 Hz), 7.01 (1H, d, J 3.7 Hz), 7.5 (1H, m), 7.84 (1H, s), 8.20 (1H, s), 8.62 (1H, s), 9.34 (1H, s), 10.59 (1H, s);

MS ^{m}/z: 347 (M+H);

Trans-isomer (176 mg, 32%), white solid. ¹H-NMR (DMSO-d₆): δ2.0 (2H, m), 3.4 (2H, broad), 4.2 (2H, m), 4.89 (1H, d, J 46.2 Hz), 6.61 (1H, dd, J 3.7, 2.2 Hz), 6.66 (1H, dd, J 12.4, 8.8 Hz), 6.75 (1H, d, J 8.8 Hz), 7.01 (1H, d, J 2.9 Hz), 7.5(1H, m), 7.84 (1H, s), 8.22 (1H, s), 8.71 (1H, s), 9.36 (1H, s), 10.46 (1H, s);

MS ^{m}/z: 347 (M+H).

### Examples 21 and 22

### Cis-and Trans-Diastereomers of 3,5′-difluoro-1-(6-cyano-3-pyridylcarboyl)-spiro[piperidine-4,2′(1H)-guinazoline]-4′-amine trifluoroacetate

Using, General Procedure E, 1-(6-cyano-3-pyridylcarbonyl)-4,4-dimethoxy-3-fluoropiperidine (375 mg, 1.28 mmol) and 2-amino-6-fluorobenzamidine dihydrochloride (271 mg, 1.20 mmol) in 2-propanol (8 mL) gave the title diastereomers.

Cis-isomer (trifluoroacetate salt; 196 mg. 33%), light yellow solid; 1H-NMR (DMSO-d₆): δ2.0 (2H, m), 3.5 (3H, m), 4.4 (1H, m), 4.64 (0.6H, d, J 45.4 Hz), 4.86 (0.4H, d, J 44.7 Hz), 6.67 (1H, dd, J 11.0, 8.8 Hz), 6.77 (1H, d, J 8.1 Hz), 7.5 (1H, m), 8.06 (1H, d, J 8.1 Hz), 8.15 (1H, d, J 8.3 Hz), 8.17 (1H, s), 8.61 (1H, s), 8.70 (1H, s), 8.80 (1H, s), 10.06 (0.4H, s), 10.14 (0.6H, s);

MS ^{m}/z: 383 (M+H).

Trans-isomer (trifluoroacetate salt: 140 mg, 24%), white solid; ¹H-NMR (DMSO-d₆): δ2.0 (2H, m), 3.6 (3H, m), 4.3 (1H, m), 4.86 (0.6H, d, J 46.5 Hz), 4.92 (0.4H, d, J 46.1 Hz), 6.6 (1H, m), 6.7 (1H, m), 7.5 (1H, m), 8.02 (1H, d, J 7.4 Hz), 8.10 (1H, d, J 8.1 Hz), 8.23 (1H, s), 8.70 (2H, s), 8.84 (1H, broad), 9.95 (0.4H, s), 10.07 (0.6H, s);

MS ^{m}/z: 383 (M+H).

### Examples 23 and 24

### Cis-and Trans-Diastereomers of 3,540 -difluoro-1-(4-methylbenzoyl)-spiro[piperidine-4,2′(1′H)-guinazoline]-4′-amine trifluoroacetate

Using General Procedure E, 4,4-dimethoxy-3-fluoro-1-(4-methylbenzoyl) piperidine (405 mg, 1.44 mmol) and 2-amino-6-fluorobenzamidine dihydrochloride (272 mg, 1.20 mmol) in 2-propanol (8 mL) gave the title diastereomers.

Cis-isomer (trifluoroacetate salt; 236 mg, 41%), light yellow solid; ¹H-NMR (DMSO-d₆): δ2.0 (2H, m), 2.31 (3H, s), 3.5 (3H, m), 4.3 (1H, m), 4.8 (1H, m), 6.68 (1H, dd, J 11.7, 8.1 Hz), 6.81 (1H, d, J 8.8 Hz), 7.25 (4H, m), 7.5 (1H, m), 8.14 (1H, s), 8.63 (1H, s), 8.79 (1H, s), 10.12 (1H, s);

MS ^{m}/z: 371 (M+H).

Trans-isomer (trifluoroacetate salt; 219 mg, 38%), white solid; ¹H-NMR (DMSO-d₆): δ2.0 (2H, m), 2.31 (3H, s), 3.4 (3H, m), 4.3 (1H, m), 4.8 (1H, m), 6.66 (1H, dd, J 11.7, 8.1 Hz), 6.74 (1H, d, J 8.1 Hz), 7.25 (4H, m), 7.5 (1H, m), 8.24 (1H, s), 8.71 (1H, s), 8.88 (1H, s), 10.01 (1H, s);

MS ^{m}/z: 371 (M+H).

### Examples 25 and 26

### Cis-and Trans-Diastereomers of 1-(6-cyano-3-pyridyicarbonyl)-3,5′, 8′-trifluorospiro[piperidine-4,2′(1′H)-guinazoline]-4′-amine trifluoroacetate

Using General Procedure F, 1-(6-cyano-3-pyridylcarbonyl)-4,4-dimethoxy-3-fluoropiperidine (1.0 g, 3.4 mmol) and 2-amino-3,6-difluorobenzamidine hydrochloride (700 mg, 3.4 mmol) in 2-propanol (20 mL) gave the title diastereomers.

Cis-isomer (free amine; 160 mg, 12%), pale yellow solid; 1H-NMR (free amine, DMSO-d₆): δ1.60-2.00 (2H, m), 3.3-3.6 (3H, m), 4.1-4.7 (2H, m), 6.1 (2H, m), 6.3 (1H, m) 6.6 (1H, bs), 7.1 (1H, m), 8.05 (1H, m), 8.15 (1H, m), 8.75 (1H, s);

MS ^{m}/z: 401 (M+H).

Trans-isomer (520 mg, 38%), pale yellow solid; ¹H-NMR (free amine, DMSO-d₆): δ1.65 (1H, m), 2.1 (1H, m), 3.0-3.9 (3H, m), 4.2-4.7 (2H, m), 6.05 (2H, m), 6.35 (1H, m), 6.6 (1H, m), 7.2 (1H, m), 8.1 (2H, ), 8.7 (1H, m);

MS ^{m}/z: 401 (M+H).

### Examples 27 to 30

### (−)-(3S,2′R)-, (+)-(3R,2′S)-, (−)-(3S,2′S)- and (+)-(3R,2′R)-Enantiomers of 1-(6-cyano-3-pyridylcarbonyl)-3,5′,8′-trifluorospiro[piperidine-4,2′(1′H )-guinazoline]-4′-amine trifluoroacetate

The cis-diastereomer (Example 26) (20 mg) was subjected to chiral HPLC with a chiral AD column (40% 2-propanol in hexanes with 0.1% diethylamine) to give the cis-(−)-(3S, 2′R)-enantioiner (8 mg, 40%) and the cis-(+)-(3R,2′S)-enantiomer (8 mg, 40%).

Cis-(−)-(3S,2′R)-enantiomer: [α]_{D}−8.4° (c 0.38, methanol): MS ^{m}/z: 401 (M+1).

Cis-(+)-(3R,2′S)-enantiomer: [α]_{D}+11.6° (c 0.38, methanol); MS ^{m}/z: 401 (M+1).

The trans-diastereomer (Example 27) (120 mg) was subjected to chiral HPLC with a chiral AD column (50% ethanolin hexanes with 0.1% diethylamine) to give the trans-(−)-(3S, 2′S)-enantiomer (25 mg, 21%) and the trans-(+)-(3R,2′R)-enantiomer (30 mg, 25%).

Trans-(−)-(3S, 2′S)-enantiomer: [α]_{D}−129° (c 0.065, methanol); MS ^{m}/z: 401 (M+1). The optical punrty is >90% ee by AD Chiral HPLC analysis.

Trans-(+)-(3R,2′R)-enantiomer: [α]_{D}+110° (c 0.31, methanol); MS ^{m}/z: 401 (M+1). The optical purity is >90% ee by AD Chiral HPLC analysis.

### Examples 31 and 32

### Cis-and Trans-Diastereomers of 1-(4-chlorobenzoyl)-3,5′,8′-trifluorospiro[piperidine-4,2′(1′H)-quinazoline]-4′-amine trifluoroacetate

Using General Procedure F, 1-(4-chlorobenzoyl)-4,4-dimethoxy-3-fluoropiperidine (79 mg, 0.31 mmol) and 2-amino-3,6-difluorobenzamidine hydrochloride (60 mg, 0.35 mmol) in 2-propanol (3 mL) gave the title diastereomers.

Cis-isomer (trifluoroacetate salt; 10 mg, 9%), pale yellow solid; ¹H-NMR (trifluoroacetate salt, DMSO-d₆) δ2.0 (2H, m), 3.2-3.7 (3H, m), 4.0-4.2 (1H, m), 4.6-4.9 (1H, m), 6.75 (1H, m), 7.4 (2H, m), 7.5 (3H, m), 7.8-8.0 (1H, m), 8.6-8.8 (1H, m), 9.9-10.2 (1H, m),

MS ^{m}/z: 409 (M+H).

Trans-isomer (trifluoroacetate salt; 33 mg, 28%), pale yellow solid; ¹H-NMR (trifluoroacetate salt, DMSO-d₆): δ1.95 (2H, m), 3.2-3.8 (3H, m), 4.4-4.9 (2H, m), 6.75 (1H, m), 7.4 (2H, m), 7.55 (3H, m), 8.2 (1H, m), 8.85 (2H, m), 10.05 (1H, m);

MS ^{m}/z: 409 (M+H).

### Examples 33 and 34

### Cis-and Trans-Diastereomers of benzyl 4′-amino-3,5′,8′-trifiuorospiro[piperidine-4,2′(1′H)-guinazoline]-1-carboxylate

Using General Procedure F, benzyl 3-fluoro-4,4-dimethoxy-1-piperidine carboxylate (5.30 g, 17.85 mmol) and 2-amino-3,6-difluorobenzamidine hydrochloride (4.08 g, 16.7 mmol) in 2-propanol (50 mL) followed by MPLC purification gave the title diastereomers.

Cis-isomer: (1.89 g, 28%). ¹H-NMR (400 MHz, CDCl₃): δ7.4 (5H, m), 6.9 (1H m), 6.3 (1H, m), 5.2 (1H, broad), 5.14 (2H, s), 4.4-4.0 (2H, m), 3.9 (1H, broad), 3.5 (2H, broad), 2.0 (1H, broad), 1.6 (3H, broad); MS ^{m}/z: 405 (M+H).

Trans-isomer: (2.90 g, 43%). MS ^{m}/z: 405 (M+H).

### Example 35

### Cis-3,5′,8′-Trifluorospiro[piperidine-4,2′(1′H)-guinazoline]-4′-amine

Benzyl cis-4′-amino-3,5′,8′-trifluorospiro[piperidine-4,2′(1′H)-quinazoline]-1-carboxylate (1.89 g, 4.67 mmol) was dissolved in methanol (20 mL) and a catalytic amount of 10% Pd/C was added. The resultin, mixture was shaken under a 40 psi hydrogen atmosphere at room temperature overnight to give the title compound (1.22 g, 96.7%).

MS ^{m}/z: 271 (M+H).

### Example 36

### Trans-3,5′,8′-Trifluorospiro[piperidine-4,2′(1′H)-guinazoline]-4′-amine

Benzyl trans-4′-amino-3,5′,8′-trifluorospiro[piperidine-4,2′(1′H)-quinazoline]-1-carboxylate (2.90 g, 7.17 mmol) was dissolved in methanol (20 mL) and a catalytic amount of 10% Pd/C was added. The resulting mixture was shaken under a 40 psi hydrogen atmosphere at room temperature overnight to give the title compound (1.89 g, 97.5%).

MS ^{m}/z: 271 (M+H).

### General Procedure G for the Synthesis of Fluoro-piperidine Spirocycles

To a solution of cis-3,5′,8′-trifluorospiro[piperidine-4,2′(1′H)-quinazoline]-4′-amine or trans-3,5′,8′-trifluorospiro[piperidine-4,2′(1′H)-quinazoline]-4′-amine (1 eq.) in dichloromethane was added triethyamine (1.5 eq.), followed by the addition of the acid chloride (1 eq.). The mixture was stirred at room temperature for 7 h, then washed with brine, dried over MgSO₄ and concentrated to dryness. The crude product was purified by C-18 column chromatography (Gilson HPLC system) using, 10 to 40% acetonitrile in water containing 0.1% trifluoroacetic acid to give the pure desired product as a trifluoroacetate salt.

### Example 37

### Cis-1-(4-Cyanobenzoyl)-3,5′,8′-trifluorosqiro[piperidine-4,2′(1′H)-guinazoline]-4′-amine trifluoroacetate

Using General Procedure G, cis-3,5′,8′-trifluorospiro[piperidine-4,2′(1′H)-quinazoline]-4′-amine (100 mg, 0.37 mmol) and 4-cyanobenzoyl chloride (61.26 mg. 0.37 mmol) in dichloromethane (5 mL) followed by HPLC purification gave the title compound (trifluoroacetate salt; 115 mg, 60.5%). ¹H NMR (CDCl₃): δ1.70˜2.03 (2H, m), 3.27˜3.46 (2H, m), 3,51˜3.80 (2H, m), 4.30˜5.0 (1H, m), 4.71 (1H, s), 5.18 (2H, s), 6.34 (1H, m), 7.01 (1H, m), 7.54 (2H, d, J 7.2 Hz), 7.71 (2H, d, J 7.2 Hz);

MS ^{m}/z: 400 (M+H).

### Example 38

Trans-1-(4-Cyanobenzoyl)-3,5′,8′-trifluorospiro[piperidine-4,2′(1′H)-guinazoline]-4′-amine trifluoroacetate

Using General Procedure G, trans-3,5′,8′-trifluorospiro[piperidine-4,2′(1′H)-quinazoline]-4′-amine (100 mg, 0.37 mmol) and 4-cyanobenzoyl chloride (61.26 mg. 0.37 mmol) in dichloromethane (5 mL) followed by HPLC purification gave the title compound (trifluoroacetate salt; 128 mg, 67.3%). ¹H NMR (CDCl₃): δ1.50˜2.23 (2H, m), 3.35˜3.80 (4H, m), 4.20˜4.62 (1H, m), 4.28 (1H, s), 5.30 (2H, bs), 6.30 (1H, m), 6.97 (1H, m). 7.52 (2H, d, J 8.0 Hz), 7.73 (2H, d, J 8.0 Hz):

MS ^{m}/z: 400 (M+H).

### Example 39

### Cis-1-(2-Furylcarbonyl)-3,5′,8′-trifluorospiro[piperidine-4,2′(1′H)-guinazoline]-4′-amine trifluoroacetate

Using General Procedure G, cis-3,5′,8′-trifluorospiro[piperidine-4,2′(1′H)-quinazoline]-4′-amine (90 mg, 0.33 mmol) and 2-furoyl chloride (43.5 mg. 0.33 mmol) in dichloromethane (5 ml) followed by HPLC purification gave the title compound (trifluoroacetate salt; 105 mg, 66.5%). ¹H NMR (CDCl₃): δ2.87 (2H, m), 3.59 (2H, br): 4.20˜4.60 (5H, m), 5.30 (1H, br); 6.29 (1H, m), 6.50 (1H, s), 6.95 (1H, m), 7.04 (1H, s), 7.26 (1H, s), 7.50 (1H, s):

MS ^{m}/z: 365 (M+H).

### Example 40

### Trans-1-(2-Furylcarbonyl)-3,5′,8′-trifluorospiro[piperidine-4,2′(1′H)-quinazoline]-4′-amine trifluoroacetate

Using General Procedure G, trans-3,5′,8′-trifluorospiro[piperidine-4,2′(1′H)-quinazoline]-4′-amine (90 mg, 0.33 mmol) and 2-furoyl chloride (43.5 mg. 0.33 mmol) in dichloromethane (5 mL) followed by HPLC purification gave the title compound (trifluoroacetate salt; 110 mg, 69.7%). ¹H NMR (trifluoroacetate salt, DMSO-d6); δ2.0 (1H, d, J 14.4 Hz), 2.41 (1H, m,), 4.34 (1H, d, J 14.4 Hz), 4.43 (1H, t, J 11.6 Hz), 4.89 (1H, d, J 46.4 Hz), 6.61 (1H, s), 6.73 (1H, m), 7.01 (1H, s), 7.53 (1H, m), 7.84 (1H, s), 8.17 (1H, s), 8.87 (1H, br), 9.19 (1H, br), 10.34 (1H, br);

MS ^{m}/z: 365 (M+H).

### Example 41

### Cis-1-(2-Thienylcarbonyl)-3,5′,8′-trifluorospiro[pipieridine-4,2′(1′H)-guinazoline]-4′-amine trifluoroacetate

Using General Procedure G, cis-3,5′,8′-trifluorospiro[piperidine-4,2′(1′H)-quinazoline]-4′-amine (90 mg, 0.33 mmol) and 2-thiophenecarbonyl chloride (48.8 mg. 0.33 mmol) in dichloromethane (5 mL) followed by HPLC purification gave the title compound (trifluoroacetate salt; 130 mg, 79.7%). ¹H NMR (trifluoroacetate salt, DMSO-d₆): δ2.04 (1H, m), 2.17 (1H, m), 3.70 (2H, m), 4.0 (2H, m), 4.74 (1H, d, J 43.2 Hz), 6.76 (1H, m), 7.08 (1H, dd, J 3.6, 4.4 Hz), 7.37 (1H, d, J 3.6 Hz), 7.53 (1H, m), 7.73 (1H, d, 4.6 Hz), 7.95 (1H, s), 8.80 (2H, br), 10.18 (1H, br);

MS ^{m}/z: 381 (M+H).

### Example 42

### Trans-1-(2-Thienylcarbonyl)-3,5′,8′-trifluorospiro[pineridine-4,2′(1′H)-quinazoline]-4′-amine trifluoroacetate

Using General Procedure G, trans-3,5′,8′-trifluorospiro[piperidine-4,2′(1′H)-quinazoline]-4′-amine (90 mg, 0.33 mmol) and 2-thiophenecarbonyl chloride (48.8 mg. 0.33 mmol) in dichloromethane (5 mL) followed by HPLC purification gave the title compound (trifluoroacetate salt; 110 mg, 67.4%). ¹H NMR (trifluoroacetate salt, DMSO-d₆): δ2.00 (1H, m), 2.40 (1H, m), 3.40 (2H, m), 4.37 (2H, m), 4.90 (1H, d, J 47.6 Hz), 6.73 (1H, m), 7.11 (1H, dd, J 3.6, 5.2 Hz), 7.38 (1H, d, J 3.6 Hz), 7.53 (1H, m), 7.76 (1H, d, J 5.2 Hz); 8.22 (1H, s), 8.69 (1H, br), 8.89 (1H, br), 9.94 (1H, br); MS ^{m}/z: 381 (M+H).

### Examples 43 and 44

### (+)-(3S,2′S)-and (−)-(3R,2′R)-Enantiomers of trans-1-(4-Cyanobenzoyl)-3,5′,8′-trifluorospiro[piperidine-4,2′(1′H)-guinazoline]-4′-amine trifluoroacetate

The trans-diastereomer of 3,5′-difluoro-1-(4-cyanobenzoyl)-spiro[piperidine-4,2′(1′H)-quinazoline]-4′-amine (Example 18, 130 mg) was passed through a chiral OD column using 15% ethanolin hexane containing 0.1% diethylamine as eluent to give the title enantiomers which were then converted into the corresponding trifluoroacetate salts.

The (+)-(3S,2′S)-enantiomer was eluted first: (66.2 mg, 39%; free amine); light yellow solid: [α]_{D}:+108.0° (methanol, c 0.13); MS ^{m}/z: 382 (M+H).

The (−)-(3R,2′R)-enantiomer was eluted second: (62.2 mg, 37%: free amine); light yellow solid; [α]_{D}:−111.4° (methanol, c 0.14); MS ^{m}/z: 382 (M+H).

### Examples 45 and 46

### (+)-(3R,2′S)- and (−)-(3S,2′R)-Enantiomers of cis-1-(4-Cyanobenzoyl)-3,5′,8′-trifluorospiro[piperidine-4,2′(1′H)-guinazoline]-4′-amine trifluoroacctate

The cis-enantiomer of 1-(4-cyanobenzoyl)-3,5′,8′-trifluorospiro[piperidine-4,2′(1′H)-quinazoline]-4′-amine (Example 37, 525 mg) was passed through an AD chiral column eluting with 20% ethanolin hexanes containing 0.1% diethylamine to give the title enantiomers which were then converted into the corresponding trifluoroacetate salts by treating with trifluoroacetic acid.

The (−)-(3S,2′R)-enantiomer was eluted first: (241 mg, 46% yield); [α]_{D}−46.4° (c 0.28, methanol). For spectroscopic data, see Example 37.

The (+)-(3R,2′S)-enantiomer was eluted second: (224 mg, 43% yield); [α]_{D}+52.8° (c 0.26, methanol). For spectroscopic data, see Example 37.

### Examples 47 and 48

### (3S,2′S)- and (3R,2′R)-Enantiomers of trans-1-(4-Cyanobenzoyl)-3,5′,8′-trifluorospiro[piperidine-4,2′(1′H)-quinazoline]-4′-amine trifluoroacetate

The trans-diastereomer of 1-(4-cyanobenzoyl)-3,5′,8′-trifluorospiro[piperidine-4,2′(1′H)-quinazoline]-4′-amine (140 mg,) was separated by an AD chiral column using 30% ethanol in hexanes containing 0.1% diethylamine as eluent.

The (3S,2′S)-enantiomer (50 mg, 34%) was eluted first, [α]_{D}−34.0° (c 1.0, CHCl₃). For spectroscopic data, see Example 38.

The (3R,2′R)-enantiomer (50 mg, 34%) was eluted second [α]_{D}+35.0° (c 1.0, CHCl₃). For spectroscopic data, see Example 38.

### Example 49

### Cis-1-(5-Cyano-2-pyridylcarbonyl)-3,5′,8′-trifluorospiro[piperidine-4,2′(1′H)-quinazoline]-4′-amine trifluoroacetate

a) 5-Cyanopicolinic Acid

A mixture of 3-cyano-6-methylpyridine (6.12 g, 51.7 mmol) and selenium dioxide (17.5 g, 157.7 mmol) in pyridine (100 mL) was heated for 10 h at 100 to 120° C. The grey solid was removed off by filtration and washed with methanol and the filtrate was evaporated to dryness. The residue was dissolved in water (150 mL) and then acidified to pH˜1 to 2 by the addition of concentrated hydrochloric acid. The precipitate was collected by filtration, washed with cold water (3×25 mL) and dried under vacuum to give a light yellow powder (6.92 ). The aqueous layer was extracted with ethyl acetate (5×200 mL). The combined organic phases were washed with saturated aqueous sodium chloride solution (3×50 mL) and dried using sodium sulphate. After filtration and concentration, a further 0.67 g of a light yellow solid was obtained. Total yield: 7.59 g (99%).

¹H-NMR (acetone-d₆): 9.08 (1H, d, J 1.9), 8.81 (1H, dd, J 8.4, 1.8), 8.30 (1H, d, J 8.3), 2.9 (1H, broad); MS (negative): 147 (100%. M−H): Purity: >95% (HPLC, C-18 column, 0 to 30% acetonitrile in water).

b) Cis-1-(5-Cyano-2-pyridylcarbonyl)-3,5′,8′-trifluorospiro[pieridine-4,2′(1H)-guinazoline]-4′-amine trifluoroacetate

A mixture of 5-cyanopicolinic acid (125 mg, 0.856 mmol) and carbonylduimidazole (138.7 mg, 0.856 mmol) in N,N-dimethylformamide (8 mL) at 0° C. was stirred for 1 h whilst being allowed to warm to room temperature. Cis-3,5′,8′-Trifluorospiro[piperidine-4,2′(1′H)-quinazoline]-4′-amine (Example 35; 243 mg, 0.899 mmol) was added in one portion and the resulting mixture was stirred at room temperature overnight. The solvent was evaporated in vacuo, the residure dissolved in ethyl acetate and the organic phase washed with brine and then dried over sodium sulfate. Evaporation and MPLC purification (silica el eluting with 10% methanolin dichloromethane containing 0.1% aqueous ammonia) gave the title compound (305 mg, 89.1%).

¹H NMR (400 MHz, DMSO-d₆): δ1.95-2.30 (2H, m), 3.50-4.20 (4H, m), 4.60-4.90 (1H, d, J 46.8 Hz), 6.70 (1H, m), 7.50 (1H, m), 7.75 (1H, m), 7.90 (1H, m), 8.40 (1H, m), 8.75 (1H, bs), 9.00-9.20 (2H, m), 10.4 (1H, bs). MS ^{m}/z: 401 (M+H).

### Examples 50 and 51

### (+)-(3R,2′S)- and (−)-(3S,2′R)-Enantiomers of cis-1-(5-Cyano-2-pyridylcarbonyl)-3,5′,8′-trifluorospiro[piperidine-4,2′(1′H)-quinazoline]-4′-amine trifluoroacetate

The cis-enantiomer of 1-(5-cyano-2-pyridylcarbonyl)-3,5′,8′-trifluorospiro[piperidine-4,2′(1′H)-quinazoline]-4′-amine (Example 49(b); 243 mg) was passed through an AD chiral column eluting with 30% ethanolin hexanes containing 0.1% diethylamine to give the title enantiomers which were then converted into the corresponding trifluoroacetate salts.

The (−)-(3S,2′R)-enantiomer (160 mg as the trifluoroacetate salt, 50% yield) was eluted first: [α]_{D}−31.7° (c 0.45, methanol).

The (+)-(3R,2′S)-enantiomer (160 mg as the trifluoroacetate salt, 50% yield) was eluted second: [α]_{D}+31.1° (c 0.6, methanol).

### Example 52

### Trans-Diastereomer of 1-(5-Cyano-2-pyridylcarbonyl)-3,5′,8′-trifluorospiro[piperidine-4,2′(1′H)-guinazoline]-4′-amine trifluoroacetate

A mixture of 5-cyanopicolinic acid (37.85 mg, 0.204 mmol) and carbonyldiumidazole (29.97 mg, 0.185 mmol) in N,N-dimethylformamide (5 mL) at 0° C. was stirred for 1 h whilst being allowed to warm to room temperature. The trans-diastereomer of 3,5′,8′-trifluorospiro[piperidine-4,2′(1′H)-quinazoline]-4′-amine (Example 36; 50 mg, 0.185 mmol) was added in one portion and the resulting mixture was stirred at room temperature overnight. The solvent was evaporated off under vacuo, the residue dissolved in ethyl acetate and the organic phase washed with brine and then dried over sodium sulfate. Evaporation and MPLC purification (silica el eluting with 5% methanolin dichloromethane containing 0.1% aqueous ammonia) gave the title product (52 mg, 70.2%).

¹H NMR (400 MHz, DMSO-d₆): δ1.85-2.10 (2H, m), 3.65-3.90 (2H, m), 4.60-4.80 (2H, m), 4.70-5.10 (1H, m), 6.70 (1H, m), 7.50 (1H, m), 7.75 (1H, m), 8.20 (1H, m), 8.40 (1H, m), 8.82 (1H, bs), 9.00 (1H, m), 9.20 (1H, bs), 10.4 (1H, bs). MS ^{m}/z: 401 (M+H).

Evaluation of Compounds for Bioloaiocal Activity

The enzyme nitric oxide synthase has a number of isofoms and compounds of formula (I), and optical isomers and racemates thereof and pharmaceutically acceptable salts thereof, may be screened for nitric oxide synthase inhibiting activity by following procedures based on those of Bredt and Snyder in Proc. Natl. Acad. Sci., 1990, 87, 682-685. Nitric oxide synthase converts ³H-L-arginine into ³H-L-citrulline which can be separated by cation exchange chromatography and quantified by scintillation counting.

In Screen for Neouronal Nitric Oxide Synthase Inhibiting Activity

The enzyme is isolated from rat hippocampus or cerebellum. The cerebellum or hippocampus of a male Sprague-Dawley rat (250-275 g) is removed following, CO₂ anaesthesia of the animal and decapitation. Cerebellar or hippocampal supematant is prepared by homogenisation in 50 mM Tris-HCl with 1 mM EDTA buffer (pH 7.2 at 25° C.) and centifuigation for 15 minutes at 20.000 g. Residual L-arginine is removed from the supernatant by chromatography through Dowex AG-50W-X8 sodium form and hydrogen form columns successively, and further centrifugation at 1000 g for 30 seconds. For the assay, 25 μl of the final supernatant is added to each of 96 wells (of a 96 well filter plate) containing either 25 μl of an assay buffer (50 mM HEPES,1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 10 minute equilibration period, 25 μl of an L-arginine solution (of concentration 18 μM ¹H-L-arginine, 96 nM ³H-L-arginine) is added to each well to initiate the reaction. The reaction is stopped after 10 minutes by addition of 200 μl of a slurry of termination buffer (20 mM HEPES, 2 mM EDTA, pH 5.5) and Dowex AG-50W-X8 200-400 mesh. Labelled L-citrulline is separated from labelled L-arginine by filtering each filter plate and 75 μl of each terminated reaction is added to 3 ml of scintillation cocktail, The L-citrulline is then quantified by scintillation counting. In a typical experiment using the cerebellar supernatant, basal activity is increased by 20,000 dpm/ml of sample above a reagent blank that has an activity of 7,000 dpm/ml. A reference standard, N-nitro-L-arginine, which gives 80% inhibition of nitric oxide synthase at a concentration of 1 μM, is tested in the assay to verify the procedure.

Screen for Human Neuronal Nitric Oxide Synthase Inhibiting Activity

Enzyme was isolated from human hippocampus, cortex or cerebellum. Cerebellar, cortical or hippocampal supernatant is prepared by homogenisation of frozen human tissue (1 to 5 g) in 50 mM Tris-HCl with 1 mM EDTA buffer (pH 7.2 at 25° C.) and centrifugation for 15 minutes at 20,000 g. Residual L-arginine is removed from the supernatant by chromatography through Dowex AG-50W-X8 sodium form and hydrogen form columns successively and further centrifugation at 1000 g for 30 seconds. Subsequently, the supernatant is passed through 2′-5 ADP Sepharose and the human nNOS eluted with NADPH,

For the assay, 25 μl of the final supematant is added to each of 96 wells (of a 96 well filter plate) containing either 25 μl of an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 30 minute equilibration period, 25 μl of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES,2 mM EDTA, pH 5.5) and Dowex AG-50W-X8 200-400 mesh.

Labelled L-citrulline is separated from labelled L-arginine by filtering each filter plate and 75 μl of each terminated reaction is added to 3 ml of scintillation cocktail, The L-citrulline is then quantified by scintillation counting.

In a typical experiment using the cerebellar supernatant, basal activity is increased by 20,000 dpm/ml of sample above a reagent blank that has an activity of 7,000 dpm/ml, A reference standard, N-nitro-L-arginine, which gives 80% inhibition of nitric oxide synthase at a concentration of 1 μM, is tested in the assay to verify the procedure.

Screen for Human Inducible Nitric Oxide Synthase Inhibiting Activity

Partially purified iNOS was prepared from cultured and lysed human DLD1 cells which had been activated with TNF-alpha, interferon gamma, and LPS. Centrifugation at 1000 g removed cellular debris and residual L-arginine was removed from the supernatant by chromatography through Dowex AG-50W-X8 sodium form and hydrogen form columns successively.

For the assay, 25 μl of the final supernatant is added to each of 96 wells (of a 96 well filter plate) containing either 25 μl of an assay buffer (50 mM HEPES,1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT. 100 μM NADPH, 10 μg/ml calmodulin, pH 7.4). Following a 30 minute equilibration period. 25 μl of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES,2 mM EDTA, pH 5.5) and Dowex AG-50W-XS 200-400 mesh.

Labelled L-citrulline is separated from labelled L-arginine by filtering each filter plate and 75 μl of each terminated reaction is added to 3 ml of scintillation cocktail, The L-citrulline is then quantified by scintillation counting.

In a typical experiment using the DLD1 supernatant, basal activity is increased by 10,000 dpm/ml of sample above a reagent blank that has an activity of 5,000 dpm/ml. A reference standard, N-methyl-L-arginine, which gives 80% inhibition of nitric oxide synthase at a concentration of 1 μM, is tested in the assay to verify the procedure.

Screen for Endothelial Nitric Oxide Synthase Inhibiting Activity

The Enzyme is isolated from human umbilical vein endothelial cells (HUVECs) by a procedure based on that of Pollock et al in Proc. Natl. Acad. Sci., 1991, 88, 10480-10484. HUVECs were purchased from Clonetics Corp (San Diego, Calif., USA) and cultured to conflueney. Cells can be maintained to passage 35-40 without significant loss of yield of nitric oxide synthase. When cells reach conflueney, they are resuspended in Dulbecco's phosphate buffered saline, centrifuged at 800 rpm for 10 minutes, and the cell pellet is then homogenised in ice-cold 50 mM Tris-HCl, 1 mM EDTA, 10% glycerol, 1 mM phenylmethylsulphonylfluoride, 2 μM leupeptin at pH 4.2. Following centrifuiation at 34,000 rpm for 60 minutes, the pellet is solubilised in the homogenisation buffer which also contains 20 mM CHAPS. After a 30 minute incubation on ice, the suspension is centrifuged at 34,000 rpm for 30 minutes. The resulting supernatant is stored at −80° C. until use.

For the assay, 25 μl of the final supernatant is added to each of 12 test tubes containing 25 μl L-arginine solution (of concentration 12 μM ¹H-L-arginine, 64 nM ³H-L-arginine) and either 25 μl of an assay buffer (50 mM HEPES,1 mM EDTA. 1.5 mM CaCl₂, pH 7.4) or 25 μl of test compound in the buffer at 22° C. To each test tube was added 25 μl of complete assay buffer (50 mM HEPES, 1 mM EDTA, 1.5 mM CaCl₂, 1 mM DTT. 100 μM NADPH, 10 μg/ml calmodulin. 12 μM tetrahydrobiopterin, pH 7.4) to initiate the reaction and the reaction is stopped after 10 minutes by addition of 2 ml of a termination buffer (20 mM HEPES,2 mM EDTA. pH 5.5).

Labelled L-citrulline is separated from labelled L-arginine by chromatography over a Dowex AG-50W-XS 200-400 mesh column. A 1 ml portion of each terminated reaction mixture is added to an individual 1 ml column and the eluent combined with that from two 1 ml distilled water washes and 16 ml of scintillation cocktail, The L-citrulline is then quantified by scintillation counting.

In a typical experiment, basal activity is increased by 5,000 dpm/ml of sample above a reagent blank that has an activity of 1500 dpm/ml, A reference standard, N-nitro-L-arginine, which gives 70-90% inhibition of nitric oxide synthetase at a concentration of 1 μM, is tested in the assay to verify the procedure.

In the screens for nitric oxide synthase inhibition activity, compound activity is expressed as IC₅₀ (the concentration of drug substance which gives 50% enzyme inhibition in the assay). IC₅₀ values for test compounds were initially estimated from the inhibiting activity of 1, 10 and 100 μM solutions of the compounds. Compounds that inhibited the enzyme by at least 50% at 10 μM were re-tested using more appropriate concentrations so that an IC₅₀ could be determined.

Model of Thermal Hyperalgesia

Compounds were tested for biological activity in a mouse model of thermal hyperalgesia (tail immersion test) following Freund's complete adjuvant-induced inflammation.

Male CD-1 mice were used (Charles River, St-Constant, Canada). Their weight was 25 to 27 g at the time of arrival, They were caged in groups of 5 in rpms thermostatically maintained at 20° C. with a 12:12 hour light/dark cycle and free access to food and water. After arrival, they were allowed to acclimatise for at least 24 hours before testing.

Injection of Freund's Complete Adjuvant (FCA)—Mice were placed in a small chamber and anesthetized using isoflurane, 5% in O₂, 800-900 ml per min. The tail of each animal was injected with 20 μl of FCA, each ml containing one mg of Mycobacterium Tuberculosis (Sigma; H 37Ra, ATCC 25177) heat killed, dried and suspended in 0.85 ml of mineral oil and 0.15 ml of mannide monooleate. The animals were allowed to wake up under observation in their home cage.

24 to 72 hours later, animals were introduced into the test room 30 minutes before the test was performed to allow them to adapt to the new environment. A 6 litre thermal bath (Lauda E100) was used for the tailimmersion test. A feedback mechanism maintained the water temperature at a fixed value throughout the testing. The expected response from the animals a flick of the tail for which a cut-off was fixed at 60 seconds.

Each group tested was composed of 10 animals. Animals were excluded if the experimenter noted the absence of inflammation in the tail or the presence of a blue tail, Sixty animals were allocated randomly to 6 groups of 10 animals. The first group was the control group and the second group was composed of the FCA injected animals. Both groups were administered the same vehicle as for the administration of the drug. Drug was administered either iv, sc or po. The other four groups were administered the drug under study dissolved in the vehicle. To control for the effect of anaesthesia administered during FCA injections, all animals tested, including control animals, were anaesthetised.

Raw data were entered into a spreadsheet software (Microsoft Excel version 1997). Information concerning the details of the experiment were added to the Excel file and stored for further analysis.

Data was collected in “seconds” which expressed the lateney to obtain a response from the animal, The mean and the standard deviation was calculated for each group and Student's T test was used to determine the statistical difference between the control group and the FCA injected group. To determine the effect of drugs, a one way analysis of variance (ANOVA) was performed, followed by a post-hoc analysis using the LSD Multiple comparison test at a 0.05 level of significanee available with the Statistica software package.

The effect of the drug under study was graphically expressed using the time difference between each dose and the FCA group, this difference being the result of the subtraction of Is the mean lateney of each dose from the mean lateney of the FCA group (i.e., Δ Lateney=Mean Lateney (Dose x)—Mean Lateney (FCA treated group+vehicle). This allowed the calculation of an ED₅₀, which is the amount of drug necessary to induce 50% of the effect. For the tail immersion test, 50% of the effect corresponds to an increase of 50% over the response lateney of the FCA treated group of animals.

Mouse Nerve Injury Mononeuropathic-induced Mechanical Allodynia

Compounds were tested for biological activity in a mouse nerve injury mononeuropathic-induced mechanical allodynia following chronic ligation of the sciatic nerve.

Male CD-1 mice were used (Charles River, St-Constant, Canada). Their weight was 25-27 g at the time of arrival. They were caged in groups of 5 in rooms thermostatically maintained at 20° C. with a 12:12 hour light/dark cycle and free access to food and water. After arrival, they were allowed to acclimatise for at least 24 hours before testing.

Chronic ligation of the sciatic nerve—Mice were anaesthetised using isoflurane (5%, 850-900 ml O₂; Aerrane/Janssen). The left hind limb of the mouse was shaved then swabbed with 70% ethanol followed by proviodine. A 1-cm incision was made along the axis of the lateral aspect of the left femur. The subcutaneous tissue was gently dissected exposing the superficial musculature. The muscles found at this location, the biceps femoris, are bisected by a line of white connective tissue. The muscle was teased apart at this junction to reveal the sciatic nerve underneath. The sciatic nerve was isolated and the tissue around it was removed. Ligation of the sciatic nerve was made by 2 ligatures (2 nods, Silk 4-0) and the skin was closed with 3M Vetbond™ surgical glue. The mice were given 6 to 8 days to recover before being tested.

Testing using the incremental von Frey filaments—Mice were placed on a wire mesh rack under a covered clear plastic cylinder measuring approximately 10-cm in diameter and 10-cm tall, A series of 7 von Frey filaments of logarithmically incremental stifffiess (0.03, 0.07, 0.17, 0.41, 1.20, 3.63 and 8.51 grams) (Stoelting) were applied to (AU-10.3) mid-plantar region of the left hind paw from beneath the mesh floor of the testing apparatus. The filaments were applied perpendicular to the plantar surface with sufficient force to cause a slight buckling against the paw, and held in place for 2 seconds. A positive response was recorded if the paw was sharply withdrawn. Flinching immediately upon removal of the filament was also considered a positive response. The starting filament is 0.41 g. If there was no response the size of the filament was increased. Otherwise, the size of the filament was decreased.

Each group tested was composed of 10 animals. Animals were excluded if the experimenter noted the absence of signs of neuropathy: slight lameness and toe flexion. Fifty animals were allocated randomly to 5 groups of 10 animals. The first group was the control group and the second group was composed of the chronic ligation treated animals. Both groups were administered the same vehicle as for the administration of the drug. The drug was administered i.v., s.c. and p.o. The other three groups were administered different concentrations of the drug under study dissolved in the vehicle. To control for the effect of anaesthesia administered during chronic ligation surgery, all animals tested, including control animals, were anaesthetised.

Data was collected in “grams” which expressed the mean amount of pressure needed to obtain a response from the animal, The mean and the standard deviation was calculated for each group and LSD Multiple comparison test was used to determine the statistical difference between the control group and the CCI treated group. To determine the effect of drugs, a one way analysis of variance (ANOVA) was performed, followed by a post-hoc analysis using the LSD Multiple comparison test at a 0.05 level of significance available with the SAS software package.

The effect of the drug under study was Graphically expressed as percent reversal between each dose and the window between the chronic ligation treated group and the control group. For comparison purposes, raw thresholds were converted to percent of maximum lo possible effect (% MPE) (according to Chaplan et al, 1994), designating vehicle treated paw withdrawal thresholds (baselines) as 0% effect, and assigning a value of 100% effect and assigning a cut-off value of 100% effect to thresholds 2.32 g: therefore, % MPE values near 100 indicate normal mechanical thresholds, whereas values near 0 indicate allodynia. This allowed the calculation of the ED 50% reversal, which is the amount of drug necessary to reverse the allodynic effect by 50% of the difference between the chronic ligation group and control level.

1. A compound according to formula (I) 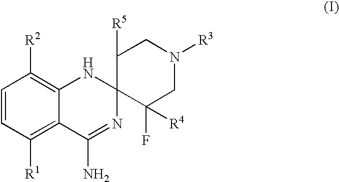
in which:
R¹ represents H, F or Cl;
R² represents H, F or CH₃;
R³ is selected from the group consisting of:
a) H; or
b) —CO—X wherein X represents:
i) a C6 to C10 aromatic ring, optionally substituted by one or more substituents selected independently from CN, Cl, F, Br, I, CF₃, OCF₃, C₁-C₃ alkyl and C₁-C₃ alkoxy;
ii) a heteroaromatic ring having from 5 to 10 ring atoms where at least one ring atom is a heteroatom selected from O, N or S; and wherein said ring is optionally substituted by one or more substituents selected independently from CN, Cl, F, Br, I, CF₃, OCF₃, C₁-C₃ alkyl and C₁-C₃ alkoxy; or
iii) C₁-C₆ alkoxy or —O—(CH₂)ₙ-phenyl, wherein n represents an integer 0 to 3;
and either both R⁴ and R⁵ represent H; or R⁴ represents H and R⁵ represents F; or R⁴ represents F and R⁵ represents H;
and diastereomers, enantiomers, racemates and tautomers thereof and pharmaceutically acceptable salts thereof.

2. A compound of formula (I), according to claim 1, wherein R⁴ and R⁵ each represents H.

3. A compound of formula (I), according to claim 1 or claim 2, wherein R¹ and R² independently represent H or F.

4. A compound of formula (I), according to claim 3, wherein R¹ represents F.

5. A compound of formula (I), according to any one of claims 1 to 4, wherein R³ represents —CO—X.

6. A compound of formula (I), according to claim 5, wherein X represents phenyl, furyl, thienyl or pyridyl optionally substituted with CN, CH₃ or Cl.

7. A compound of formula (I), according to claim 1, which is:
cis-1-(4-cyanobenzoyl)-3-fluorospiro[piperidine-4,2′(1′H)-quinazoline]-4′-amine;
trans-1-(4-cyanobenzoyl)-3-fluorospiro[piperidine-4,2′(1′H)-quinazoline]-4′-amine;
cis-1-(4-chlorobenzoyl)-3-fluorospiro[piperidine-4,2′(1′H)-quinazoline]-4′-amine;
trans-1-(4-chlorobenzoyl)-3-fluorospiro[piperidine-4,2′(1′H)-quinazoline]-4′-amine;
cis-1-(6-cyano-3-pyridylcarbonyl)-3-fluorospiro[piperidine-4,2′(1′H)-quinazoline]-4′-amine;
trans-1-(6-cyano-3-pyridylcarbonyl)-3-fluorospiro[piperidine-4,2′(1′H)-quinazoline]-4′-amine;
cis-3-fluoro-1-(4-methylbenzoyl)-spiro[piperidine-4,2′(1′H)-quinazoline]-4′-amine;
trans-3-fluoro-1-(4-methylbenzoyl)-spiro[piperidine-4,2′(1′H)-quinazoline]-4′-amine;
cis-3-fluoro-1-(2-furylcarbonyl)-spiro[piperidine-4,2′(1′H)-quinazoline]-4′-amine;
trans-3-fluoro-1-(2-furylcarbonyl)-spiro[piperidine-4,2′(1′H)-quinazoline]-4′-amine;
cis-3-fluoro-1-(2-thienylcarbonyl)-spiro[piperidine-4,2′(1′H)-quinazoline]-4′-amine;
trans-3-fluoro-1-(2-thienylcarbonyl)-spiro[piperidine-4,2′(1′H)-quinazoline]-4′-amine;
cis-3,5′-difluoro-1-(2-thienylcarbonyl)-spiro[piperidine-4,2′(1′H)-quinazoline]-4′-amine;
trans-3,5′-difluoro-1-(2-thienylcarbonyl)-spiro[piperidine-4,2′(1′H)-quinazoline]-4′-amine;
cis-3,5′-difluoro-1-(4-chlorobenzoyl)-spiro[piperidine-4,2′(1′H)-quinazoline]-4′-amine;
trans-3,5′-difluoro-1-(4-chlorobenzoyl)-spiro[piperidine-4,2′(1′H)-quinazoline]-4′-amine;
cis-3,5′-difluoro-1-(4-cyanobenzoyl)-spiro[piperidine-4,2′(1′H)-quinazoline]-4′-amine;
trans-3,5′-difluoro-1-(4-cyanobenzoyl)-spiro[piperidine-4,2′(1′H)-quinazoline]-4′-amine;
cis-3,5′-difluoro-1-(2-furylcarbonyl)-spiro[piperidine-4,2′(1′H)-quinazoline]-4′-amine;
trans-3,5′-difluoro-1-(2-furylcarbonyl)-spiro[piperidine-4,2′(1′H)-quinazoline]-4′-amine;
cis-3,5′-difluoro-1-(6-cyano-3-pyridylcarbonyl)-spiro[piperidine-4,2′(1′H)-quinazoline]-4′-amine;
trans-3,5′-difluoro-1-(6-cyano-3-pyridylcarbonyl)-spiro[piperidine-4,2′(1′H)-quinazoline]-4′-amine;
cis-3,5′-difluoro-1-(4-methylbenzoyl)-spiro[piperidine-4,2′(1′H)-quinazoline]-4′-amine;
trans-3,5′-difluoro-1-(4-methylbenzoyl)-spiro[piperidine-4,2′(1′H)-quinazoline]-4′-amine;
cis-1-(6-cyano-3-pyridylcarbonyl)-3,5′,8′-trifluorospiro[piperidine-4,2′(1′H)-quinazoline]-4′-amine;
(−)-(3S,2′R)-1-(6-cyano-3-pyridylcarbonyl)-3,5′,8′-trifluorospiro[piperidine-4,2′(1′H)-quinazoline]-4′-amine;
(+)-(3R,2′S)-1-(6-cyano-3-pyridylcarbonyl)-3,5′,8′-trifluorospiro[piperidine-4,2′(1′H)-quinazoline]-4′-amine;
trans-1-(6-cyano-3-pyridylcarbonyl)-3,5′,8′-trifluorospiro[piperidine-4,2′(1′H)-quinazoline]-4′-amine;
(−)-(3S,2′S)-1-(6-cyano-3-pyridylcarbonyl)-3,5′,8′-trifluorospiro[piperidine-4,2′(1′H)-quinazoline]-4′-amine;
(+)-(3R,2′R)-1-(6-cyano-3-pyridylcarbonyl)-3,5′,8′-trifluorospiro[piperidine-4,2′(1′H)-quinazoline]-4′-amine;
cis-1-(4-chlorobenzoyl)-3,5′,8′-trifluorospiro[piperidine-4,2′(1′H)-quinazoline]-4′-amine;
trans-1-(4-chlorobenzoyl)-3,5′,8′-trifluorospiro[piperidine-4,2′(1′H)-quinazoline]-4′-amine;
benzyl cis-4′-amino-3,5′,8′-trifluorospiro[piperidine-4,2′(1′H)-quinazoline]-1-carboxylate;
benzyl trans-4′-amino-3,5′,8′-trifluorospiro[piperidine-4,2′(1′H)-quinazoline]-1-carboxylate;
cis-3,5′,8′-trifluorospiro[piperidine-4,2′(1′H)-quinazoline]-4′-amine;
trans-3,5′,8′-trifluorospiro[piperidine-4,2′(1′H)-quinazoline]-4′-amine;
cis-1-(4-cyanobenzoyl)-3,5′,8′-trifluorospiro[piperidine-4,2′(1′H)-quinazoline]-4′-amine;
trans-1-(4-cyanobenzoyl)-3,5′,8′-trifluorospiro[piperidine-4,2′(1′H)-quinazoline]-4′-amine;
cis-1-(2-furylcarbonyl)-3,5′,8′-trifluorospiro[piperidine-4,2′(1′H)-quinazoline]-4′-amine;
tans-1-(2-furylcarbonyl)-3,5′,8′-trifluorospiro[piperidine-4,2′(1′H)-quinazoline]-4′-amine;
cis-1-(2-thienylcarbonyl)-3,5′,8′-trifluorospiro[piperidine-4,2′(1′H)-quinazoline]-4′-amine;
trans-1-(2-thienylcarbonyl)-3,5′,8′-trifluorospiro[piperidine-4,2′(1′H)-quinazoline]-4′-amine;
(+)-(3S,2′S)-trans-1-(4-cyanobenzoyl)-3,5′,8′-trifluorospiro[piperidine-4,2′(1′H)-quinazoline]-4′-amine;
(−)-(3R,2′R)-trans-1-(4-cyanobenzoyl)-3,5′,8′-trifluorospiro[piperidine-4,2′(1′H)-quinazoline]-4′-amine;
(+)-(3R,2′S)-cis-1-(4-cyanobenzoyl)-3,5′,8′-trifluorospiro[piperidine-4,2′(1′H)-quinazoline]-4′-amine;
(−)-(3S,2′R)-cis-1-(4-cyanobenzoyl)-3,5′,8′-trifluorospiro[piperidine-4,2′(1′H)-quinazoline]-4′-amine;
(3S,2′S)-trans-1-(4-cyanobenzoyl)-3,5′,8′-trifluorospiro[piperidine-4,2′(1′H)-quinazoline]-4′-amine;
(3R,2′R)-trans-1-(4-cyanobenzoyl)-3,5′,8′-trifluorospiro[piperidine-4,2′(1′H)-quinazoline]-4′-amine;
cis-1-(5-cyano-2-pyridylcarbonyl)-3,5′,8′-trifluorospiro[piperidine-4,2′(1′H)-quinazoline]-4′-amine;
(+)-(3R,2′S)-cis-1-(5-cyano-2-pyridylcarbonyl)-3,5′,8′-trifluorospiro[piperidine-4,2′(1′H)-quinazoline]-4′-amine;
(−)-(3S,2′R)-cis-1-(5-cyano-2-pyridylcarbonyl)-3,5′,8′-trifluorospiro[piperidine-4,2′(1′H)-quinazoline]-4′-amine;
trans-1-(5-cyano-2-pyridylcarbonyl)-3,5′,8′-trifluorospiro[piperidine-4,2′(1′H)-quinazoline]-4′-amine;
and acid addition salts thereof.

8. A compound of formula (I), as defined in any one of claims 1 to 7, for use in therapy.

9. A pharmaceutical formulation comprising a compound of formula (I), as defined in any one of claims 1 to 7, or an optical isomer, racemate or tautomer thereof or a pharmaceutically acceptable salt thereof, optionally in admixture with a pharmaceutically acceptable diluent or carrier.

10. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of human diseases or conditions in which inhibition of nitric oxide synthase activity is beneficial.

11. The use as claimed in claim 10 wherein it is predominantly the inducible isoform of nitric oxide synthase that is inhibited.

12. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of pain.

13. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of inflammation.

14. A method of treating, or reducing the risk of, a human disease or condition in which inhibition of nitric oxide synthase activity is beneficial which comprises administering to a person suffering from or susceptible to such a disease or condition, a therapeutically effective amount of a compound of formula (I), as defined in any one of claims 1 to 7, or an optical isomer, racemate or tautomer thereof or a pharmaceutically acceptable salt thereof.

15. A method of treatment according to claim 14 in which it is predominantly the indicible isoform of nitric oxide synthase that is inhibited.

16. A method of treating, or reducing the risk of pain, which comprises administering to a person suffering from or susceptible to such a condition a therapeutically effective amount of a compound of formula (I), as defined in any one of claims 1 to 7, or an optical isomer, racemate or tautomer thereof or a pharmaceutically acceptable salt thereof.

17. A method of treating, or reducing the risk of inflammation, which comprises administering to a person suffering from or susceptible to such a condition a therapeutically effective amount of a compound of formula (I), as defined in any one of claims 1 to 7, or an optical isomer, racemate or tautomer thereof or a pharmaceutically acceptable salt thereof.

18. A process for the preparation of a compound of formula (I), as defined in any one of claims 1 to 7, and optical isomers, racemates and tautomers thereof and pharmaceutically salts thereof, which comprises preparing a compound of formula (I) by:
(a) reacting a corresponding compound of formula (II) or a salt thereof 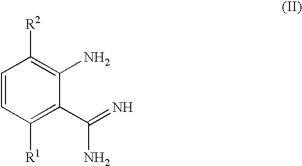
wherein R¹ and R² are as defined in claim 1,
with a compound of formula (III) or a salt thereof 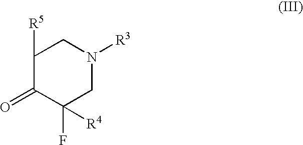
wherein R³, R⁴ and R⁵ are as defined in claim 1; or
(b) reacting a corresponding compound of formula (II) or a salt thereof,
with a compound of formula (IV) or a salt thereof 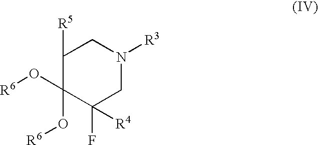
wherein R³, R⁴ and R⁵ are as defined in claim 1 and R⁶ represents C₁-C₃ alkyl; or
(c) reacting a corresponding compound of formula (V) or a salt thereof, 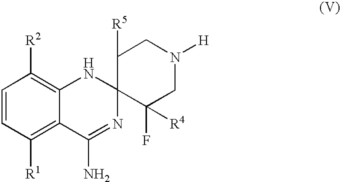
wherein R¹, R², R⁴ and R⁵ are as defined in claim 1;
with a compound of formula L—CO—X wherein X is as defined in claim 1 and L represents a leaving group such as Cl or OH;
and where desired or necessary converting the resultant compound of formula (I), or another salt thereof, into a pharmaceutically acceptable salt thereof; or converting the resultant compound of formula (I) into a further compound of formula (I); and where desired converting the resultant compound of formula (I) into an optical isomer thereof.

19. An intermediate useful in the synthesis of a compound of formula (I), according to claim 1, said intermediate being a compound of formula (III) 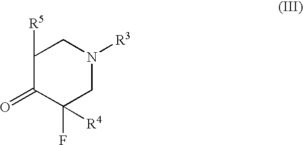
wherein R³, R⁴ and R⁵ are as defined in claim 1,
with the proviso that the compound wherein R⁴ and R⁵ each represent H and R³ represents —CO—O tert-butyl is disclaimed.

20. An intermediate useful in the synthesis of a compound of formula (I), according to claim 1, said intermediate being a compound of formula (IV) 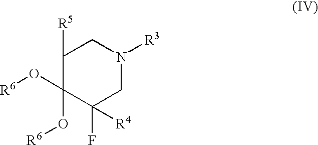
wherein R³, R⁴ and R⁵ are as defined in claim 1 and R⁶ represents C₁-C₃ alkyl.

21. A process for the preparation of a compound of formula (VII): 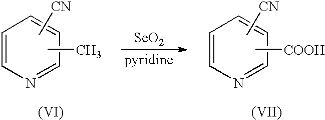
wherein a corresponding compound of formula (VI) is oxidised by heating with selenium dioxide in pyridine, generally at about 100° C.
